(19) United States
(12) Patent Application Publication
(10) Pub. No.: US 2004/0115172 A1
(43) Pub. Date: Jun. 17, 2004

(54) Assembled implant, including mixed-composition segment

(75) Inventors: John R. Bianchi, Gville, FL (US); C. Randall Mills, Town of Tioga, FL (US); P.J. Gorham, La Crosse, FL (US); Michael Esch, Warwick, NY (US); Kevin C. Carter, Gainesville, FL (US); Diane Carter, Gainesville, FL (US); Pat Coleman, Gainesville, FL (US); Kevin Ross, Gainesville, FL (US); Harry W. Rambo, Alachua, FL (US); Darren G. Jones, Gainesville, FL (US); Dayna Buskirk, Gainesville, FL (US); Russell S. Donda, Gainesville, FL (US)

Correspondence Address: DONALD J. POCHOPIEN, ESQ., McANDREWS, HELD & MALLOY, LTD., 34TH FLOOR, 500 WEST MADISON STREET, CHICAGO, IL, 60661, US

(73) Assignee: Regeneration Technologies, Inc.

(21) Appl. No.: 10/387,322

(22) Filed: Dec. 23, 2002

Related U.S. Application Data
(63) Continuation of application No. 09/941,154, filed on Aug. 27, 2001.
Application No. 09/941,154 is a continuation of application No. 09/782,594, filed on Feb. 12, 2001.
Application No. 09/941,154 is a continuation-in-part of application No. 09/378,527, filed on Aug. 20, 1999, now Pat. No. 6,652,818.
Application No. 09/378,527 is a continuation-in-part of application No. 09/191,232, filed on Nov. 13, 1998, now Pat. No. 6,482,584.
Application No. 09/941,154 is a continuation of application No. 09/370,194, filed on Aug. 9, 1999, now Pat. No. 6,223,534.
Application No. 09/941,154 is a continuation of application No. 29/123,227, filed on May 12, 2000, now Pat. No. D461248.
Application No. 09/941,154 is a continuation of application No. 09/528,034, filed on Mar. 17, 2000.
Application No. 09/528,034 is a continuation of application No. 09/481,319, filed on Jan. 11, 2000, now Pat. No. 6,497,726.
Application No. 09/528,034 is a continuation of application No. 09/363,909, filed on Jul. 28, 1999, now abandoned.
(60) Provisional application No. 60/181,622, filed on Feb. 10, 2000.

(51) Int. Cl. A61K 45/00

(52) U.S. Cl. 424/93.7

(57) ABSTRACT

This invention provides a method for manufacture of autograft, allograft and xenograft implants which comprises assembling such implants from smaller pieces of graft materials to form a larger graft implant product. One segment of an assembled graft implant is comprised of two or more discrete regions having distinct characteristics and/or properties.

### CROSS REFERENCE TO RELATED APPLICATION

This application is a continuation of U.S. application Ser. No. 09/782,594, filed Feb. 12, 2001, pending, which is a continuation-in-part of provisional application serial No. 60/181,622, filed Feb. 10, 2000, and of U.S. application Ser. No. 09/378,527, filed on Aug. 20, 1999, pending, which is a continuation in part of U.S. application Ser. No. 09/191,232, filed on Nov. 13, 1998, pending; and of U.S. application Ser. No. 09/390,194, filed on Sep. 7, 1999, pending; and of U.S. application Ser. No. 29/123,227, filed May 12, 2000, pending, and of U.S. application Ser. No. 09/528,034, filed Mar. 17, 2000, which is a continuation of U.S. application Ser. No. 09/481,319, filed Jan. 11, 2000; and of U.S. patent application Ser. No. 09/363,909, filed Jul. 28, 1999, and of copending application Ser. No. 09/905,683, filed Sep. 16, 2001, which is a continuation of copending application Ser. No. 09/701,933, filed Aug. 25, 1998, which is a continuation in part of 08,920,630, abandoned, filed Aug. 27, 1997; the priority and benefit of which are claimed herein under 35 U.S.C. Sections 119, and 120. All of these applications are incorporated by reference.

### FIELD OF THE INVENTION

This invention relates to implants and methods for their preparation wherein components of the implant are assembled from constituent pieces to produce a complete implant. An implant according to this invention comprises two or more segments comprised of mineralized, or demineralized bone segments or a segment comprising both demineralized and mineralized regions juxtaposed to one another.

### BACKGROUND OF THE INVENTION

In the field of medicine, there has been an increasing need to develop implant materials for correction of biological defects. Particularly in the field of orthopedic medicine, there has been the need to replace or correct bone, ligament and tendon defects or injuries. As a result, there have emerged a number of synthetic implant materials, including but not limited to metallic implant materials and devices, devices composed in whole or in part from polymeric substances, as well as allograft, autograft, and xenograft implants. It is generally recognized that for implant materials to be acceptable, they must be pathogen-free, and must be biologically acceptable. Generally, it is preferable if the implant materials may be remodeled over time such that autogenous bone replaces the implant materials. This goal is best achieved by utilizing autograft bone from a first site for implantation into a second site. However, use of autograft materials is attended by the significant disadvantage that a second site of morbidity must be created to harvest autograft for implantation into a first diseased or injured site. As a result, allograft and xenograft implants have been given increasing attention in recent years. However, use of such materials has the disadvantage that human allograft materials are frequently low in availability and are high in cost of recovery, treatment and preparation for implantation. By contrast, while xenograft implant materials, such as bovine bone, may be of ready availability, immunological and disease transmission considerations imply significant constraints on the ready use of such materials.

In view of the foregoing considerations, it remains the case that there has been a long felt need for unlimited supplies of biologically acceptable implant materials for repair of bone and other defects or injuries. This invention provides a significant advance in the art, and largely meets this need, by providing materials and methods for production of essentially any form of implant from component parts to produce assembled implants. In particular, the invention is directed to compositions, methods and kits that relate to an implant, in which at least one single segment is demineralized or comprises a combination of mineralized and demineralized regions. Among the advantages of this invention are the benefits in strength, structural support, and flexibility, depending on the particular implant and its use in a patient in need thereof.

In addition, reference is made herein to U.S. Pat. No. 5,899,939 to Boyce, which issued on May 4, 1999, the disclosure of which is hereby incorporated by reference as if fully set forth herein.

Finally, reference is made herein to U.S. Pat. No. 6,025,538 to Yaccarino, which issued on Feb. 15, 2000, the disclosure of which is hereby incorporated by reference as if fully set forth herein.

The present invention advances the art beyond the references cited above by disclosing and claiming implants that comprise a combination of mineralized and demineralized regions provided in a single segment (discrete piece), which is distinguishable from that disclosed in U.S. Pat. No. 6,200,347 (teaching homogenous demineralization of a single segment). The importance of demineralized bone in implants is described in U.S. Pat. No. 6,090,998, and U.S. patent application Ser. Nos. 09/417,401, 09/518,000, 09/585,772, and 09/778,046, all assigned to the assignee of the present invention, and all of which are incorporated by reference.

### SUMMARY OF THE INVENTION

This invention provides a method for manufacture of allograft, allograft and xenograft implants which comprises assembling such implants from smaller pieces of graft materials to form a larger graft implant product. Some pieces of such graft materials for assembly are demineralized, and are combined with other pieces of graft materials that are mineralized.

Accordingly, it is one object of this invention to provide a method for assembly of multiple bone implant shapes from smaller bone implant pieces.

Another object of this invention is to provide assembled bone implants. Related to this object is the object of assembling components of an assembled allograft in such a way as to compensate for disproportionate shrinkage among components during freeze drying so as to still obtain precision interference fits.

Another object of this invention is to provide a method whereby otherwise wasted tissue may be used in the production of useful orthopedic implants.

Another object of this invention is to provide an implant having a combination of at least one region that is demineralized, juxtaposed to at least one region that is mineralized. Another object of this invention is to combine a segment of an implant having combination of mineralized and demineralized regions with other segments that are mineralized.

Further objects and advantages of this invention will be appreciated from a review of the complete disclosure and the appended claims.

### BRIEF DESCRIPTION OF THE DRAWINGS

Attached to this invention disclosure are a large number of sketches which demonstrate a wide variety of assembled implants which may be prepared and used according to this invention.

FIG. 1 is a flow chart showing the formation of various sub-component parts of an assembled implant according to this invention, from which assembled implants and a kit comprising these parts may be formed according to the disclosure of this invention.

FIG. 2 provides a schematic of an assembled implant according to this invention.

FIG. 3 provides a schematic of an assembled implant according to this invention.

FIGS. 4-7 provides a schematic of an assembled implant according to this invention.

FIGS. 8-9 provides a schematic of an assembled implant according to this invention.

FIGS. 10-14 provides a schematic of an assembled implant according to this invention.

FIGS. 15-18 provides a schematic of an assembled implant according to this invention.

FIG. 19 provides a schematic of an assembled implant according to this invention.

FIG. 20 provides a schematic of an assembled implant according to this invention.

FIG. 21 provides a schematic of an assembled implant according to this invention.

FIG. 22 provides a schematic of an assembled implant according to this invention.

FIG. 23 shows the assembly of a dowel from component pieces.

FIG. 24 shows the reinforcement of an implant using a cortical bone pin.

FIG. 25 shows the reinforcement of an implant using a cortical bone pin and a cortical bone disc.

FIG. 26 shows the reinforcement of cancellous bone implants using a plurality of cortical bone pins.

FIG. 27 shows the formation of an assembled implant comprising soft and hard tissues.

FIG. 28 shows a segment comprising a central mineralized region and demineralized regions.

FIG. 29 shows the arrangement of the segment of FIG. 28 positioned between two mineralized implant segments.

FIG. 30 shows an alternative embodiment comprising more than one segment having mineralized and demineralized regions.

FIG. 31 shows an embodiment of the subject assembled implant supported by a scaffold.

FIG. 32 shows an additional embodiment comprising segments fastened together through a friction fit.

FIG. 33 shows a two-segment assembled implant fastened together through a friction fit.

FIG. 34 shows an embodiment that comprises two segments that interlock together in a transverse cross-over configuration.

### DETAILED DESCRIPTION OF THE PREFERRED EMBODIMENTS

Currently, autograft, allograft and xenograft products are produced as solid, continuous materials. For example, bone dowels (see U.S. Pat. No. 5,814,084, hereby incorporated by reference), Smith-Robinson cervical spine implants, iliac crest grafts, and the like are harvested and machined from single, continuous pieces of bone. The present invention provides methods for manufacture of autograft, allograft and xenograft implants by assembling such implants from smaller pieces of graft materials to form a larger graft implant product. As a result, increased utilization of valuable implant materials is achieved, thereby more effectively meeting the ever-increasing demands for graft implant materials. In addition, greater flexibility is achieved in the types and shapes of implant materials is achieved. Essentially, any implant piece that may be required may be formed according to the present invention, and orthopedic surgeons may be provided with kits of assemblable parts which may be formed in the course of a surgical procedure to precisely meet the needs of a given patient or procedure. In yet another aspect of this invention, existing graft products may be strengthened or reinforced by assembly of different types of graft materials into an assembled product. One example of such a reinforced product is a cancellous wedge, block, dowel or the like into which is inserted reinforcing pins of cortical bone. As a result, those skilled in the art will understand from this disclosure that different sections of tissue may be assembled to make a complete graft implant. Furthermore, this invention provides for the product of assembled implants comprising any one or combinations of allograft materials, autograft materials, xenograft materials, synthetic materials, metallic materials and the like. Furthermore, the assembled implants or the component pieces which are combined to form the assembled implant may be pre-treated or treated after assembly to incorporate any desired biologically active or inert materials. Thus, for example, in an assembled bone dowel implant according to this invention, the assembled bone dowel comprises segments of cortical bone pinned to each other by means of cortical bone pins. Prior to assembly or after assembly, the graft materials are soaked, infused, impregnated, coated or otherwise treated with bone morphogenetic proteins (BMP's), antibiotics, growth factors, nucleic acids, peptides, and the like.

It is also noted that the compositions and structures disclosed and claimed herein may be obtained from allograft, xenograft or autograft sources, and are comprised of cortical, cancellous, or cortico-cancellous types of bone tissue, or combinations thereof. As disclosed herein, the compositions and structures disclosed and claimed herein are comprised of mineralized hone, demineralized bone, or combinations thereof. Also, a preferred pre-treatment is to subject allograft and xenograft-sourced bone material to one of the cleansing processes described in U.S. patent application Ser. No. 09/363,909, filed Jul. 28, 1999, the related PCT application serial number PCT/US00/20629, filed Jul. 28, 2000 and published as WO01/08715A1, and U.S. patent application Ser. No. 09/191,232, filed Nov. 13, 1998.

In essence, one method to reduce antigenicity (disclosed in U.S. Ser. No. 09/363,909 and WO01/08715A1) is to treat bone material in hydrogen peroxide, or hydrogen peroxide in combination with a detergent such as Triton X-100 or Sodium Dodecyl Sulfate (SDS), or another chaotropic agent, such as urea, guanidinium hydrochloride, Tween, TNBP, and mixtures of these agents. This is followed by contacting with a defatting solvent, such as acetone, isopropanol, hexane, or combinations of these. The primary object is to remove the non-collagenous protein from bone graft materials, and thereby reduce antigenicity.

In another method, disclosed in U.S. Ser. No. 09/191,232, efficient cleaning and passivation (inactivation of pathogens) is achieved by sequential depressurization and pressurization of a chamber containing bone graft materials, where these materials are being exposed to cleaning/chaotropic solutions and solvents including those described above. This process has been found to improve penetration of the cleaning solutions. Thus, for the bone compositions and structures disclosed and claimed herein, these pre-treatments may be applied to clean and reduce antigenicity of the finished materials.

It will be appreciated that variously shaped wafers, blocks, rings, washer-shaped bone pieces and the like may be affixed to each other in any secure and biologically acceptable manner. Preferably, the assembled pieces of bone are affixed to each other by means of pins, screws, rods, interference fit, threaded fits, key-way fit, and the like made from cortical bone. These fixation pieces are machined in a CNC lathe or the like to appropriate dimensions and are then threaded into mating holes tapped in the pieces to be assembled, or are pressed into drilled holes through adjacent pieces to be assembled by a pneumatic press or the like. In this fashion, very strong and tightly fitted pieces of implant materials may be joined and implanted. The assembled pieces may first be machined to desired dimensions and shapes, prior to assembly, the assembled implant may be machined, or both.

As noted above, the implant according to this invention may comprise an assembled cancellous block, dowel or the like, harvested from the iliac crest or another suitable site. As is known in the art, due to the wafer-like structure of cancellous bone, such grafts have low load-bearing characteristics. There exist reports in the literature of instances of extrusion, expulsion or collapse of iliac crest wedges, Cloward Dowels, and the like when utilized, for example, in spinal fusions. Nonetheless, use of cancellous bone is preferable over use of cortical bone implants, since cancellous bone is more osteoconductive than cortical bone. According to this invention, a Cloward Dowel, iliac crest wedge, or cancellous bone block, dowel or the like is reinforced by insertion therein of cortical bone pins. According to the method of this invention, cortical implants may also be reinforced by insertion therein of cortical bone pins, including when an assembled implant is prepared comprising different segments of cortical bone, cancellous bone or both. Insertion of the reinforcing pins provides an implant with multiple load-bearing pillars. The pins may be made to protrude from the surface of the implant to engage with inferior, superior or both surfaces of bone between which the implant is inserted. Thus, in a spinal implant, pin protrusions may be employed to created contact between the implant and the vertebral bodies, thus preventing extrusion and reinforcing a secure fit of the implant between adjacent vertebrae. We have, surprisingly, found that cortical pins of about 4.5 mm in diameter may each support a load of up about 2700 newtons (160 Mpa). Thus, according to the method of this invention, multiple pins may be inserted into an implant to produce a load-bearing capacity of known proportions (e.g. 10,000 newtons by insertion of five pins).

A further advantage of this invention is that it permits use of tissues that are not currently amenable to standard autograft, allograft or xenograft harvesting and processing procedures, such as ribs, metatarsal bone and the like. In addition, useful implant materials may be harvested and produced from otherwise un-useable donor tissues. In addition, due to the different nature of various segments of bone that are incorporated into the assembled, reinforced implants of this invention, various shaping methods aside from CNC lathe or other known procedures may be applied to different segments of the implant. Thus, a cancellous portion of bone implant may be compression molded, and then affixed to other portions of cortical or cancellous bone machined according to different or similar principles. In addition, due to the ability provided by this invention to assemble implant pieces, implants of unusual sizes and dimensions may be prepared and machined. Thus, implants of 100 mm in size could be machined, for example, for corpectomies, when otherwise bone stock for manufacture of such implant dimensions would not be available.

In view of the present disclosure, it will be appreciated that this invention provides a wide variety of assembled implants and implant parts: dowel shaped implants comprising assembled dowel segments, between about two to about ten segments, pinned together by one or more cortical bone pins. The assembled segments may closely abut each other or may be spread apart from each other. Such implants may be prepared by harvesting discs of cortical bone, drilling and optionally tapping holes therein, and inserting shafts of cortical pins therethrough, or therein, optionally by threading portions thereof for torquing into optionally tapped holes. The thus produced dowels may be tapered or have parallel sides. In addition, dowels which are harvested as a cross-section across the intramedullary canal of a long bone, as in U.S. Pat. No. 5,814,084, which might otherwise not pass production specifications, due to penetration of one outside wall into the intramedullary canal, may be completed by insertion therein of a cortical pin. Likewise, where a sidewall is otherwise considered to be too narrow, a “doughnut” of bone may be affixed to the sidewall by means of a cortical pin. A longer dowel may be prepared by affixing two dowels to each other. A posterior longitudinal interbody fusion implant (PLIF) may be machined from a single piece of cortical bone, or be assembled from two pieces of bone which are affixed to each other by means of a cortical pin. A bone screw may also be prepared according to the method of this invention by affixing multiple pieces of cortical bone to each other with a cortical bone pin and then machining a thread on the exterior of the assembled bone pieces. It will further be appreciated from this disclosure that different portions of the assembled implant may be demineralized, partially or fully, to achieve a level of elasticity or compressibility not otherwise present in cortical or cancellous bone. Specific embodiments of assembled implants having a combination of demineralized and mineralized regions, present or assembled into a single discrete piece (i.e., a segment), are shown to possess superior properties. Different portions of bone may also be retained on a shaft by means of a cotter-pin type device.

According to one embodiment, a segment is mineralized allograft bone, and this region may be intimately contacted on two sides by two regions of partially demineralized allograft bone. Demineralized regions of a single segment may be formed according to conventional methods, such as by dipping a portion of a segment of mineralized allograft bone in a demineralizing acidic solution to demineralize that portion while leaving the adjacent portion mineralized. Alternately, a segment of a larger assembled implant comprising both mineralized and demineralized regions may be formed and later joined together (such as by biocompatible adhesives, bone pastes, tongue and groove, etc.) into a structure that is or can be divided (such as cut transversely) into a number of segments and subsequently assembled. The term “demineralized” is well known in the art, and for the purposes of this invention is defined to be the removal of minerals, such as by dissolution in acid, from a material such as bone.

As used herein, a “mixed-composition segment” is defined to describe a segment of an allograft implant that is comprised of two or more regions having different characteristics and/or properties. For example, a mixed-composition segment can comprise a region comprising demineralized bone or mineralized bone attached to another region comprising a synthetic material. Also, it is noted that “demineralized,” when not preceded by either “partially” or “fully,” is taken to include, subject to the specific context, both partially and fully demineralized. Also, when referring to a particular mixed-composition segment, the segment may be described as a “demineralized bone segment comprising a region of mineralized bone,” and this is taken to mean a segment that has at least one region of mineralized bone and at least one region of demineralized bone.

In addition to assembled implants, instruments may be conveniently prepared according to the methods of this invention which may be utilized for insertion of other implants. In one embodiment of this invention, therefore, an implant driver is produced wherein the driving mechanism itself is formed from assembled cortical pins which protrude into mating recesses in an implant device. The instrument may be torqued to adequate loads to induce implantation of spinal implants and the like.

In developing the various embodiments of the present invention, one technical issue of merit is the need to develop a process whereby donor tissue, whether hard or soft tissue, allograft or xenograft tissue, may be treated in such a fashion as to eliminate the possibility of cross contamination between tissue segments obtained from different sources. While it is possible to practice the present invention to advantage using tissue obtained from a single screened donor, the real economies of scale and commercially viable application of the present technology is best realized by implementation of an efficient and reliable tissue decontamination process. Ideally, the process is one which permits multiple segments of soft or hard tissue to be treated simultaneously so that a stock of materials for assemblage of implants according to the present invention is facilitated. Accordingly, on preferred method for treatment of tissue, disclosed in PCT publication WO 00/29037, the disclosure of which is hereby incorporated herein by reference as if fully set forth herein (and priority of the US Patent filings which gave rise to this application is hereby claimed for that purpose). Accordingly, in this aspect of the invention, a process is claimed whereby an assembled allograft or xenograft tissue implant is prepared by treating the tissue in a closed container in which different cleaning solutions are contacted with the implant segments, either before or after assembly and machining into the final implant form, either in the presence or absence of sonication, with rapid oscillation of pressure in the closed container, to achieve deep cleaning and interpenetration of cleaning solvents into the interstices of porous implants or tissues. Solutions including, but not limited to detergent solutions, peroxide solutions and the like are used in such procedure, and terminal sterilization with gamma irradiation, gaseous sterilants known in the art or other terminal sterilization procedures known in the art are employed to ensure safe implantation of the assembled implants according to this invention.

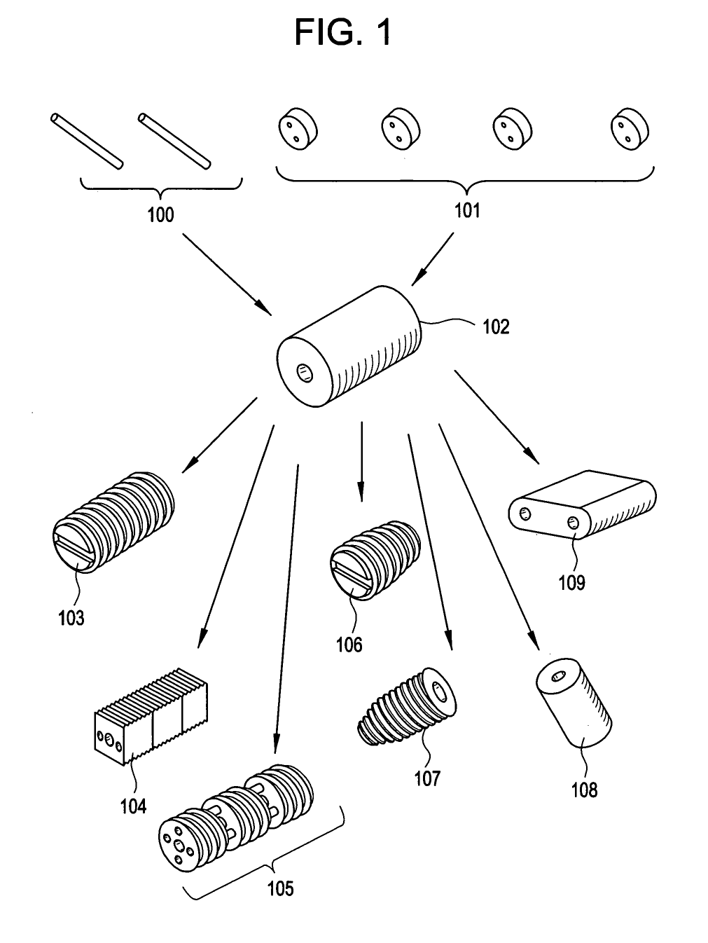

Referring now to FIG. 1, there is shown a flow-chart representing various elements that may be processed and assembled according to this invention. Cortical bone pins 100 are used to assemble a series of bone discs 101 into a pre-part 102 which is then machined into a series of final products: Threaded dowels, 103; small blocks 104; unique shapes, 105 such as a “wedding-cake” like shape wherein discs bearing threads are spaced apart from each other leaving voids 105′ into which additional materials may be inserted, with the discs retained in fixed relation to each other by means of the through pins 100; tapered dowels 106; screws 107; smooth cylinders 108; or large blocks 109. From this figure, it will be appreciated that a central concept relevant to the present invention is the ability to machine smaller parts of tissue, specifically bone tissue, such as cortical bone, cancellous bone, cortical-cancellous bone, portions of which may be demineralized (see, for example, U.S. Pat. No. 6,090,998, hereby incorporated herein by reference for this purpose), and assemble these portions of tissue using, preferably, cortical bone pins. The assembled tissue pieces may be machined prior to assembly, and then, upon assembly, a complete implant is ready for implantation. Alternatively, the tissue pieces may first be assembled, and the assembled pieces may then be machined into any desired final form. The order of assembly and machining will be determined by the specific forms of implant required for a particular application. In FIG. 1, a series of pre-machined tissue forms are disclosed, which may conveniently be included in a kit for use as needed by an orthopedic surgeon. Thus, for example, where a particular implant of specific dimensions is required, the surgeon is able to select pre-shaped implant segments to fill a particular geometric space and shape in the spine of an implant recipient. Numerous permutations and combinations of implant pieces for assembly are possible, based on the pre-machined assemblable implant pieces included in such a kit, and those skilled in the art will appreciate that the skilled orthopedic surgeon will be able to create implants as needed when supplied with such a kit. Thus, a preferred kit includes discs of bone, cortical bone, cancellous bone, allograft or xenograft, also referred to herein as “washers” or “doughnuts” such that a center hole is provided for press-fitting or screwing on of the discs to a cortical bone or synthetic or metallic shaft or pin. The discs may be demineralized, mineralized, or partially demineralized. Also desirable in such a kit are plugs of cortical bone, cancellous bone, or cortical-cancellous bone, including at least one through hole, and optionally more than one such through hole, for insertion of pins therethrough. Ovals, squares, rectangles and irregular shapes may also be provided in certain kits for specific applications. It will further be appreciated, based on the present disclosure, that inclusion of a bone paste, such as that disclosed in WO99/38543, hereby incorporated by reference, may be beneficial for filling any voids that remain, and to implant with the assembled implant, osteogenic material, (i.e. osteoconductive material, Osteoinductive material, or both, as well as material that assists in adhering the implant to the site of implantation). Further, a molded implant may be combined with the assembled implant of this invention. A preferred molded implant for orthopedic applications is disclosed in PCT publication WO 00/54821, the disclosure of which is hereby incorporated by reference.

It is noted that assembled allografts may be assembled at and distributed from a central location, or, as discussed above, assembled around the time of surgery to meet a specific requirement of a patient in need thereof. In many applications it is desirable to have a tight and accurate interference fit between cortical bone pins and the holes in bone pieces that are connected by the bone pin. The target range for such an interference fit is 0.001 to 0.003 inches (e.g., the pin diameter is 0.001 to 0.003 inches larger than the hole diameter, and is pressed fit into place). However, it has been learned that freeze-drying the pins and other bone pieces exerts a disproportionate shrinkage upon the pins compared to the hole diameters. That is, the pin shrinks slightly more than the hole. Uncorrected, this would result in a less accurate, and less acceptable, interference fit.

The following method has been adopted to solve this problem. A bone pin, preferably of cortical bone, of a desired diameter is vacuum dried for at least five hours. This drying is preferably at room temperature and at a negative pressure of approximately 100 milliTorre. This pre-treatment results in a shrinkage of approximately 80 percent of the total shrinkage that would occur in freeze drying. The pin diameter is measured, and a hole is made in the discs (or other shapes that are to be assembled) using an appropriately sized reamer. The target size for the hole is 0.002 to 0.0025 inches smaller than the post-vacuum drying pin diameter. Preferably, prior to this drilling the discs or other shapes have been kept saturated with moisture to maintain a consistent size and subsequent shrinkage percent. After all holes are drilled, the pin(s) and discs or other shapes are assembled, and then freeze dried. The resulting assembled allografts have been found to have interference fits in the desired target range. This method is applicable to the various embodiments described in this disclosure. Alternatively, where segments are provided in a kit for assembly prior to surgery, the discs and pins are preferably freeze-dried as disassembled. After freeze-drying, the diameter of the pins is measured and the appropriate size hole is made in the disc. This allows the provision of multiple parts in a kit, wherein the parts can be assembled together such that the requisite friction is acheived to keep the parts securely together.

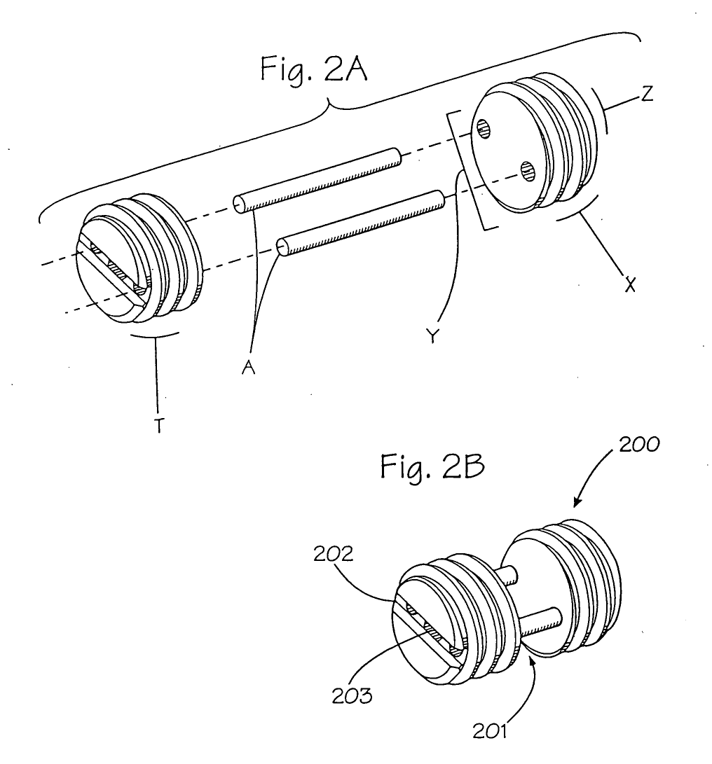

With reference to FIG. 2, there is shown two machined bone pieces, T and Z each of which bear external threading X and holes Y into which pins A are inserted to form the assembled graft 200. As can be seen, the assembled graft 200 comprises a void, 201 into which osteogenic material may be inserted prior to or after implantation. The pins Y may be metal pins, but preferably are pins machined from cortical bone. This enables the entire implant to remodel into autogenous tissue over time, such as vertebral bone, when the implant 200 is inserted into the intervertebral space. The graft 201 is also shown with a groove, 202 in which a driver may be inserted to provide rotational torque for insertion of the implant. An instrument attachment hole, 203, is also provided, to ensure that the implant remains securely on the head of the driver means in the process of surgical implantation. Naturally, those skilled in the art will appreciate that the segments Z and T may be brought into close abutment with each other, thereby eliminating the space 201. In that event, the length of the pins A would be modified to prevent unnecessary protrusion, although in some applications, protrusion may be useful when driving the implant 200 into place. It will also be appreciated that the number of pins used, while represented as two in this figure, may be fewer or more in number, depending on the particular application, the extent of torsional or compressive loads, and the like anticipated to be experienced by the implant once in situ. In some applications, the insertion of reinforcing cortical bone pins establishes a pillar structure such that two or more cortical bone pins are load-bearing. This application allows the use of materials in the segments that do not initially bear a substantial load, that load being born by the cortical bone pin pillars, and these materials have the opportunity to reform into bone that will provide subsequent structural load-bearing.

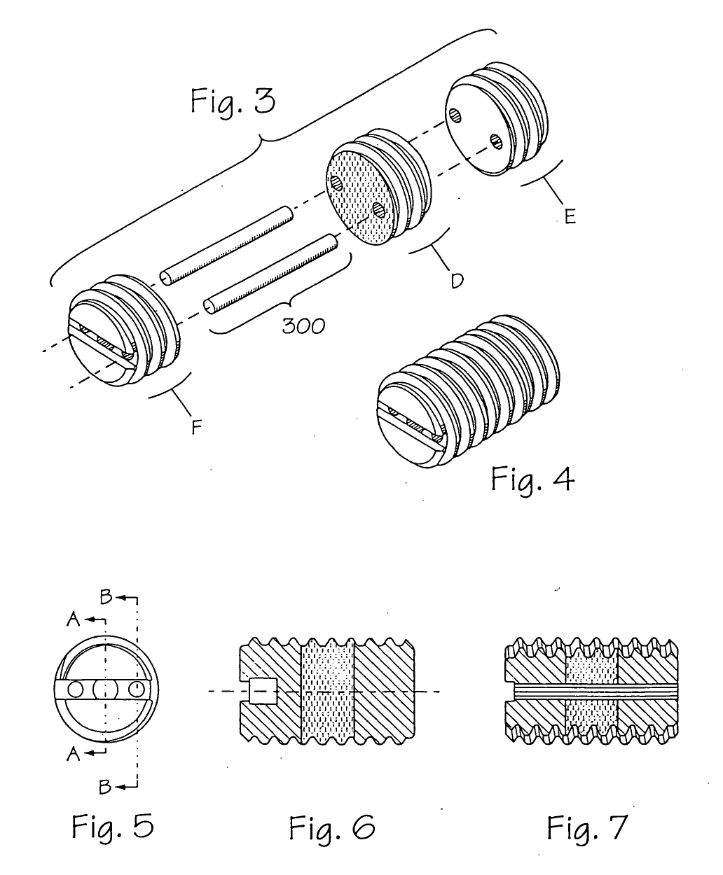

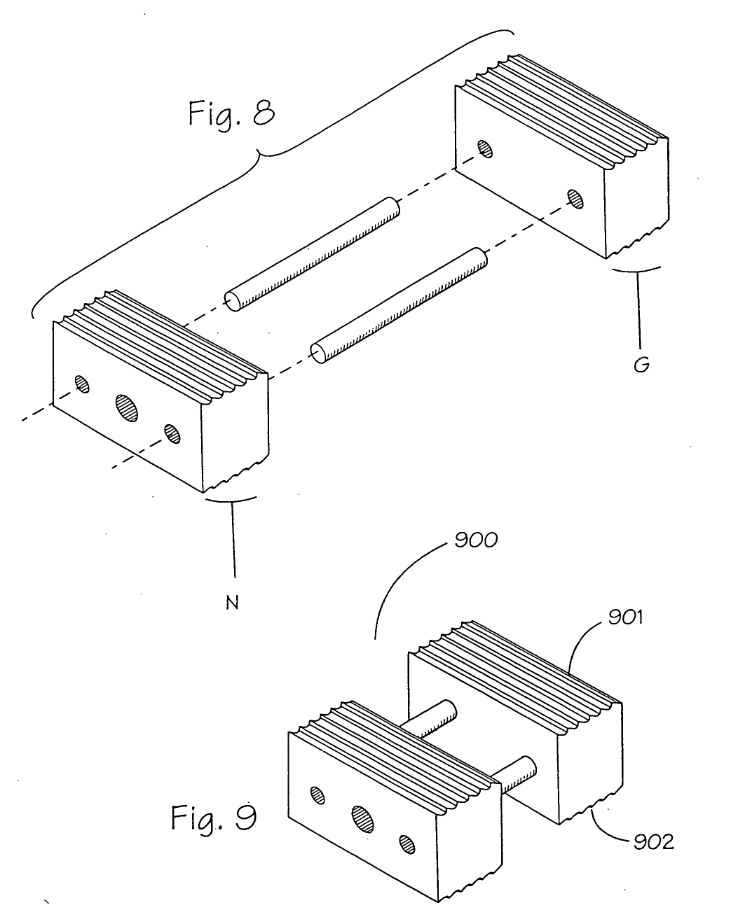

FIG. 3 shows an implant assembled from three principal segments F, D, and E, which are held together by pins 300. In this implant, the waffle-shaped structure of implant segment D is intended to represent the use of cancellous bone, which is abutted on either side by cortical bone, which forms segments F and E. The fully assembled implant is shown in FIG. 4, while FIGS. 5, 6 and 7 show end-on views, and cross sectional views A-A and B-B, respectively. Those skilled in the art will appreciate from this disclosure that segment F, segment D, or segment E may be demineralized according to methods known in the art. Likewise, all of these segments maybe demineralized. Where a flexible implant is required, the implant may be assembled, and the entire implant may be demineralized. Where flexibility is important in one dimension and structural support is also required, one solution is to have one or more segments of an composite bone graft be a mixed-composition segment which comprises at least one mineralized region and at least one demineralized region (described in detail below).

FIG. 8 shows an embodiment of this invention wherein rectangular bone segments N and G are assembled into implant 900, shown in FIG. 9. Features 901 and 902 which comprises ridges, teeth, or other external features are machined into the superior and inferior faces of the implants in order to assist in retention of the implants once placed in situ.

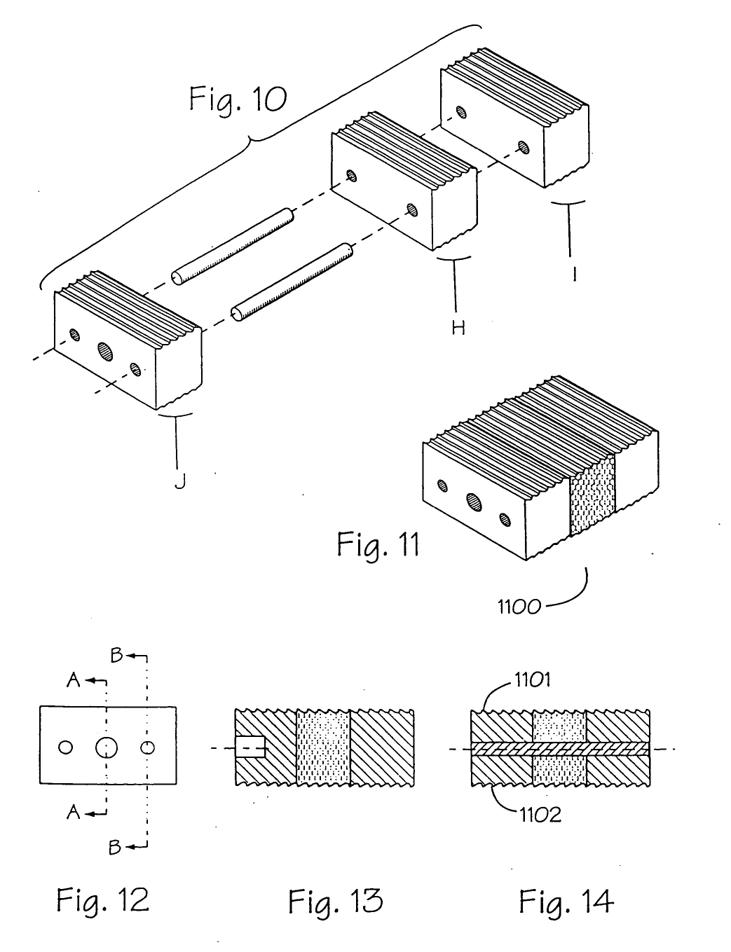

FIGS. 10-14 show the assembly of elements J, H, and I into implant 1100, shown end-on, in cross-section A-A and B-B, in FIGS. 12-14, respectively. As can be seen, bone element H is shown with a waffle-like structure to represent that this element may be cancellous bone, demineralized bone, a polymer composite, such as poly-L-Lactic acid, polyglycolic acid, or the like. Features 1101 and 1102 represent external grooves or teeth machined into the superior and inferior surfaces of the implant to assist in retention of the implant once placed in situ.

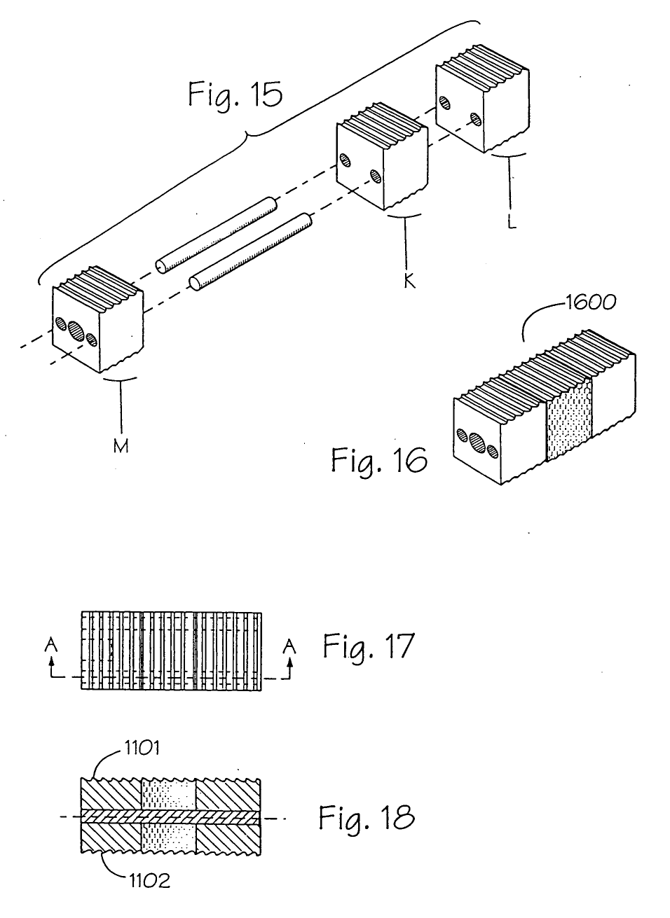

FIGS. 15-18 show the assembly of elements M, K, and L, each of which is a substantially cubic bone element, using pins 1500. FIG. 17 is a top view, showing cross section A-A, represented in FIG. 18, with the final assembled implant 1600 shown in FIG. 16.

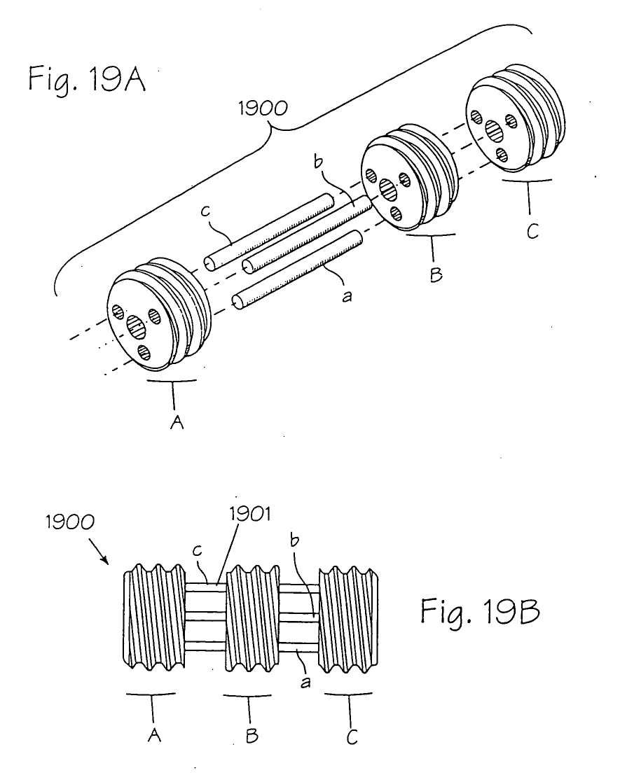

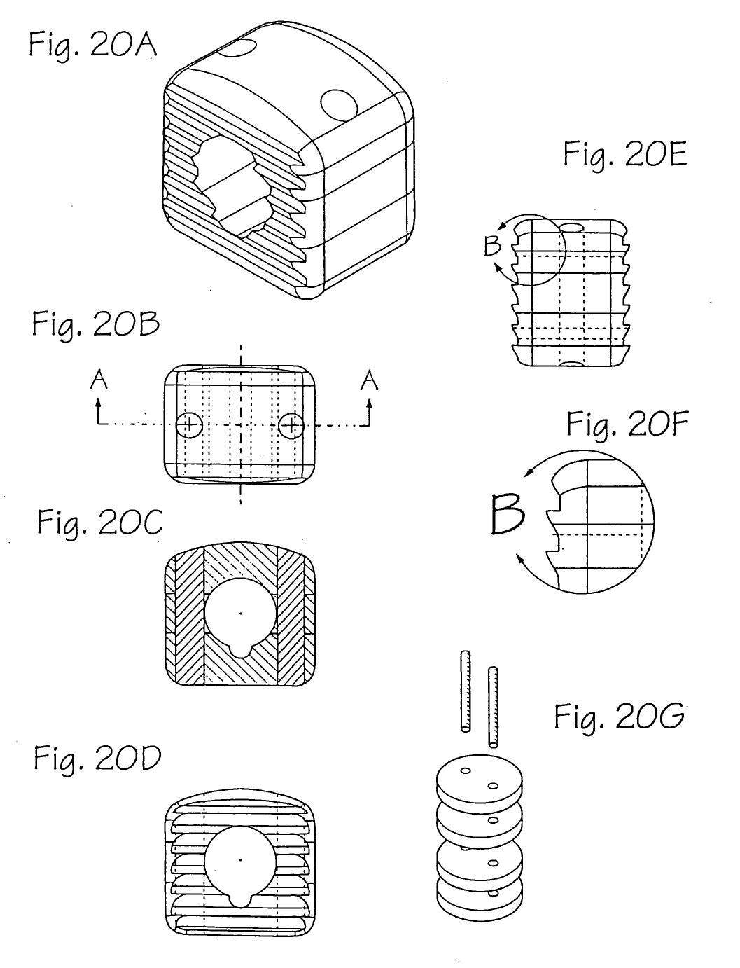

FIG. 19 shows a “Wedding-Cake” design of an implant 1900 assembled from units A-C, pinned together by pins a-c. Void area 1901 is available for filling with osteogenic materials.

FIG. 20 shows implant 2000 which is an assembled Cervical Smith Robinson implant similar to that shown in PCT publication WO99/09914, hereby incorporated by reference. This implant is fashioned from a series of assembled bone pieces 2001 and machined into the desired final shape.

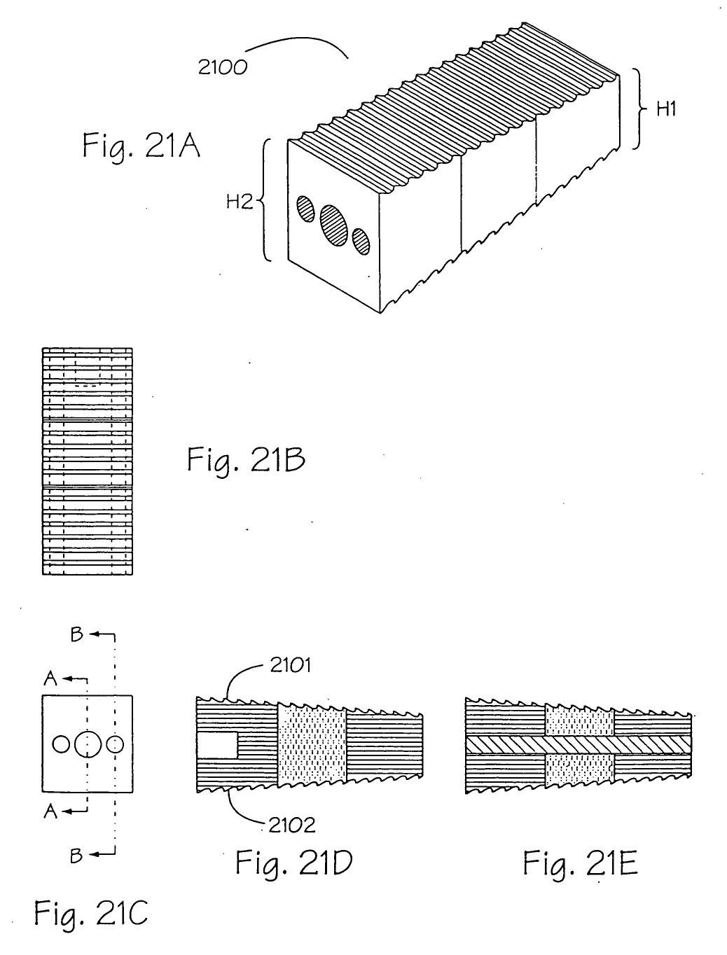

FIG. 21 shows implant 2100 assembled from two cortical bone pieces and one cancellous bone piece, and pinned together. The implant has an anterior height H1 which is smaller than posterior height H2, which permits retention of correct spinal lordosis upon implantation, for example, in a posterior lumbar intervertebral implant fixation procedure. Superior and inferior features 2101, 2102 prevent expulsion of the implant once place in situ.

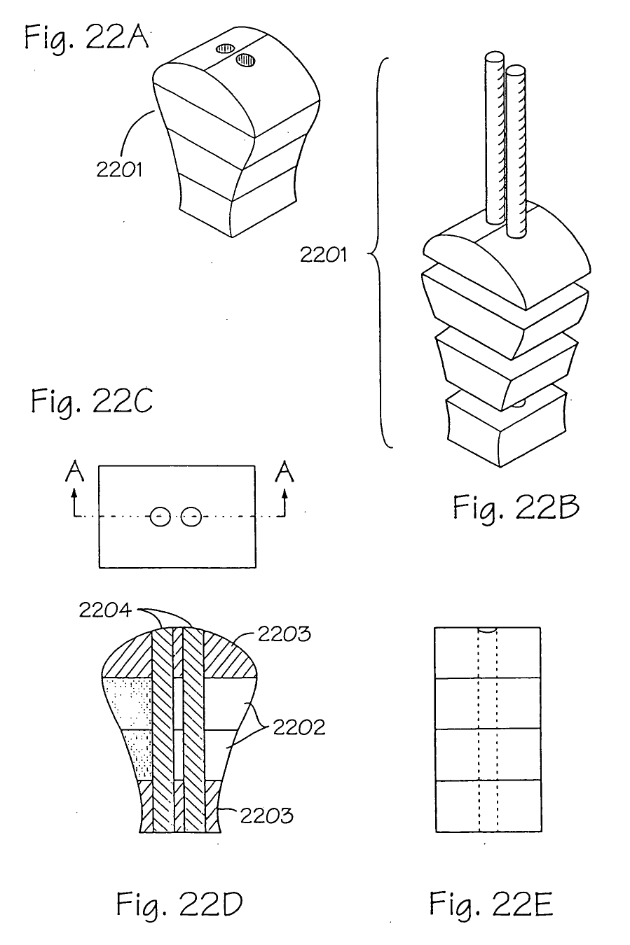

FIG. 22 shows an implant 2200 assembled from a series of sub-implant pieces 2201. The implant may contain cancellous bone 2202 segments, as well as cortical bone 2203 segments and cortical bone pins 2204.

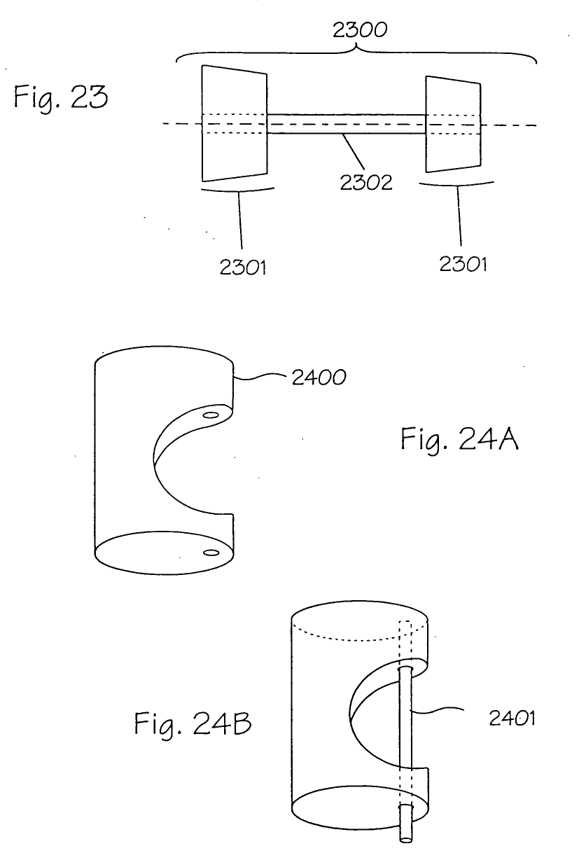

FIG. 23 shows the formation of a tapered dowel 2300 by assembling “doughnut” or “disc” or “washer” shaped bone pieces 2301 on a cortical bone shaft 2302 by using washer pieces of differing diameter. This figure only shows two discs, but a continuous dowel is formed by using discs of a graded diameter between each end of the cortical bone shaft 2302. In FIG. 24, FIG. 24A shows a bone dowel in which one sidewall of a bone dowel 2400 such as that disclosed and claimed in U.S. Pat. No. 5,814,084, hereby incorporated by reference, is “out of specifications” due to being too narrow or absent. This is repaired in FIG. 24B according to this embodiment of the invention by incorporation of an allograft or xenograft cortical bone pin 2401, to form a complete bone dowel. In this manner, valuable biological material which might otherwise be unusable for a particular application may be salvaged for use by employing the methodology of this invention.

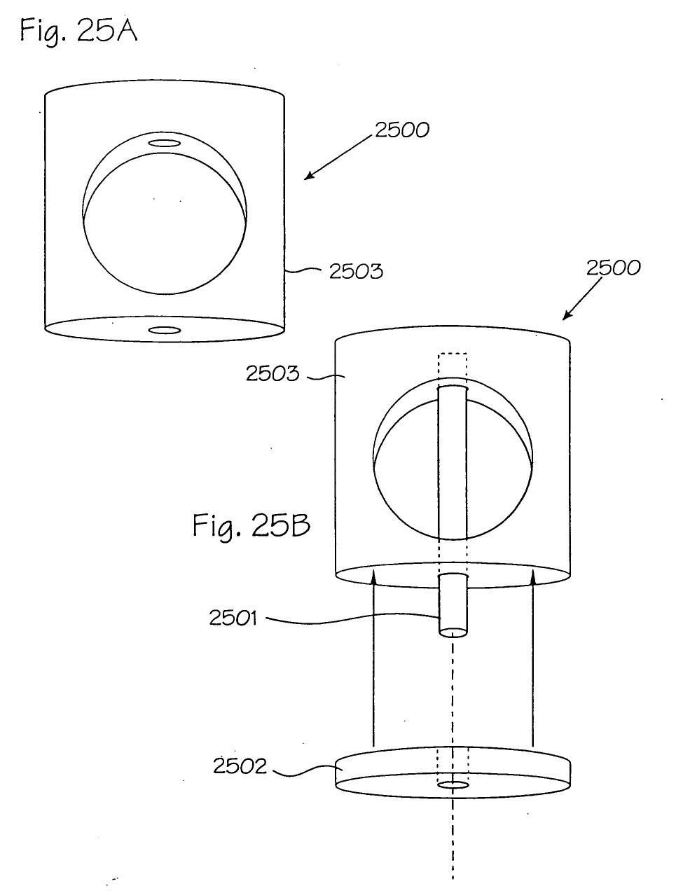

In FIG. 25, a similar procedure for salvaging a dowel 2500 is shown whereby a pin 2501 is driven through the center of the dowel 2500 to reinforce the dowel longitudinally. Furthermore, where an endcap 2503 of the dowel is “out of spec” for being too narrow, the endcap is reinforced by press-fitting a cortical bone disc 2502 onto the end of the pin 2501.

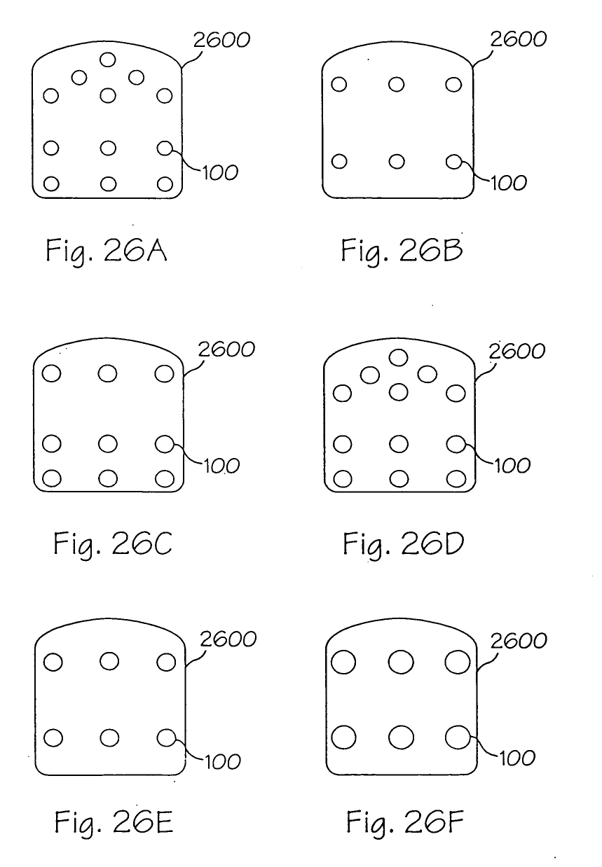

In FIG. 26, a series of cancellous bone implants 2600 are reinforced by inclusion therein of a series of cortical pins 100. Each cortical pin of a 2 mm diameter has been found to support approximately 2000 newtons of axial compressive load. Accordingly, cancellous bone implants of essentially any desired height and compressive strength may be assembled in this manner by affixing several layers of cancellous bone with cortical bone pins. Naturally, based on this disclosure, those skilled in the art will appreciate that other materials may be included in such a “sandwich” of bone materials. The cancellous bone may be soaked in a solution containing growth factors, such as, but not limited to, bone morphogenetic proteins, fibroblast growth factors, platelet derived growth factor, cartilage derived morphogenetic proteins, stem cells, such as mesenchymal stem cells, osteoprogenitor cells, antibiotics, antiinflammatory compounds, anti-neoplastic compounds, nucleic acids, peptides, and the like. Those skilled in the art will also appreciate that layers of cortical bone may be included, layers of biocompatible synthetic polymers and the like may also be included in the stacked bone implant. Various shapes may also be built upon, using for example, circles, ellipses, squares, and the like, as necessary for a given application.

In a further aspect of the present invention, the assembled implant is driven by cortical pins to seat in an implant site, using a driver that engages cortical bone pins with purchase sites on the implant. Thus, for example, not meant to be limiting, the driver may comprise a handle with projecting cortical pins which engage with holes in the assembled allograft, thereby providing a site for torquing the implant into position.

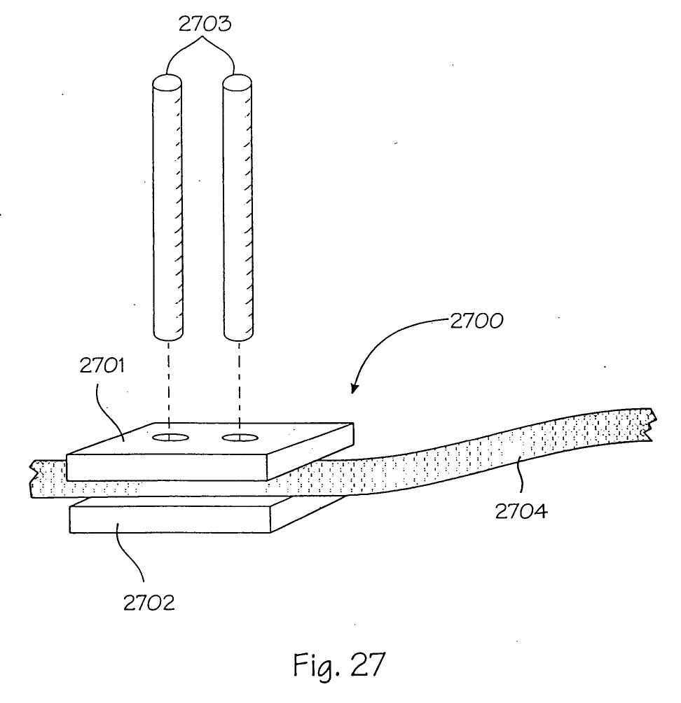

In a further embodiment according to this invention, assembled cortical bone blocks, or cortical cancellous bone blocks, or bone blocks comprised of a combination of cortical bone, cortico-cancellous bone, cancellous bone, and/or synthetic materials as described elsewhere herein, are assembled in combination with wedged or pinned soft tissue, such as tendon, ligament, skin, collagen sheets, or the like, to create grafts similar to naturally occurring tissue sites, such as the bone-tendon interface found at the patella. Such combination implants permit reconstruction of sites such as the Anterior Cruciate Ligament (ACL) or Posterior Cruciate Ligament (PCL). According to one embodiment of the invention, a ligament or tendon or skin or collagen sheet membrane is pinned between adjacent blocks of cortical bone. Accordingly, various implants, such as known bone-tendon-bone implants which are in short supply may be supplanted by assemblage of an implant comprising assembled bone blocks, between which is fixed a ligamentous tissue, including but not limited to ligament, tendon, demineralized bone, and the like Referring to FIG. 27, there is shown one example of this embodiment of the present invention in which an implant 2700 is assembled from a superior bone block 2701, an inferior bone block 2702 and a wedged flexible tissue, such as a ligament or tendon or portion of demineralized bone 2704, all of which are pinned together with cortical bone pins 2703 or other fixation means. The superior bone block, 2701, is comprised of three segments of bone, 2701a-c, pinned together by pin 2715. Naturally, those skilled in the art will appreciate, based on this disclosure, that other shapes of bone blocks, such as rounded bone blocks, and other types of combinations of soft and hard tissues may be assembled according to this disclosure. However the example of such an implant 2700 may be used instead of having to harvest a bone-tendon-bone implant from cadaveric knees, which tissue is in short supply.

Another variation of this embodiment is to construct a bone-tendon-bone type of implant that is comprised of at least one block made from substantially synthetic materials, attached to a tendon-like section of an allograft, autograft or xenograft sourced ligament, tendon, skin or collagen. Still another variation is to construct a bone-tendon-bone type of implant that is comprised of a synthetic tendon-like material, attached to a block at one or both ends, where the block is comprised of allograft, autograft or xenograft bone, and the block is a single piece or a multi-segment assembled bone graft. Examples of synthetic materials, not meant to be limiting, are biocompatible materials selected from the group consisting of nylon, polycarbonate, polypropylene, polyacetal, polyethylene oxide and its copolymers, polyvinylpyrolidone, polyacrylates, polyesters, polysulfone, polylactide, poly(L-lactide) (PLLA), poly(D,L-lactide) (PLA), poly(glycolide) (PGA), poly(L-lactide-co-D,L-Lactide) (PLLA/PLA), poly(L-lactide-co-glycolide) (PLA/PGA), poly(glocolide-co-trimethylene carbonate) (PGA/PTMC), polydioxanone (PDS), polycaprolactone (PCL), polyhydroxybutyrate (PHBT), poly(phosphazenes), poly(D,L-lactide-co-caprolactone) (PLA/PCL), poly(glycolide-co-caprolactone) (PGA/PCL), poly(phosphase ester), polyanhydrides, polyvinyl alcohol, and hydrophilic polyurethanes. These materials, some of which are bioaborbable, can be used in combination with one another to form the synthetic section of the graft.

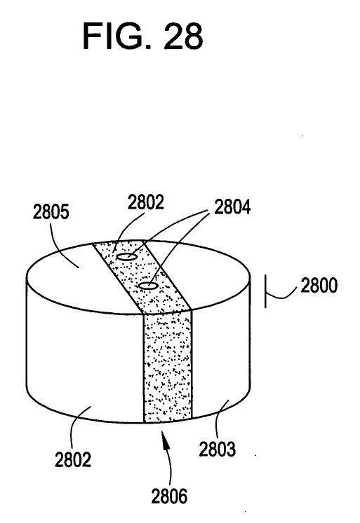

Another aspect of the invention is an allograft segment wherein at least one region is mineralized, and at least one region is demineralized. For example, FIG. 28 depicts an allograft unit, 2800, that has a central mineralized region, 2801, with two demineralized regions, 2802 and 2803, one to either side of the mineralized region. Two holes, 2804, pass from the top, 2805, to the bottom, 2806, of the allograft segment, 2800. As described above, these holes, 2804, are used for assembly of this segment with other segments, as by passing pins, dowels, or other attachment means through the holes to connect two or more allograft segments in a line.

One method of producing this segment is to start with a fully mineralized piece of allograft of the shape depicted in FIG. 28. One side, such as that represented in FIG. 28 as 2802, is subjected to an appropriate acid demineralizing regime (such as described earlier in this application) until it is to a desired level of demineralization for the purpose of the allograft segment, 2800. Then the opposing side, 2803, is similarly subjected to said regime. The regions exposed to the demineralizing regime are immersed in suitable solutions to remove acids and other components that may be toxic, inflammatory, or inhibitive of cellular infiltration. The middle mineralized region does not contact the acid solution of the regime. The resultant segment is referred to as a mixed-composition segment (“MCS”). As described below, this may be combined with other segments to form an assembled allograft.

The demineralizing regime is varied depending on the desired results. In one example, a central demineralized area is produced by blocking the outside surfaces (sides and top and bottom surfaces near the sides) of a cylinder of bone, allowing acid solution exposure only to a central circular area. In this and in other exposure regimes, a transition zone of demineralization may exist between the target area (subject to demineralization) and the blocked area (designed to remain mineralized), in which the degree of mineralization changes from the exposed demineralized region to the non-exposed mineralized region. The extent of the transition zone can vary, and can be adjusted to some extent by the demineralization regime to better meet a particular application for the implant.

An alternative means of producing an allograft segment such as 2800 is to prepare one or more demineralized regions and assemble them with one or more mineralized regions. The assembly would be secured together by means previously described. This is referred to as an assembled mixed-composition segment (“AMCS”), which may be further combined with other segments to form a larger assembled allograft.

It is noted that the degree of demineralization spans a broad range, with increased exposure to acid (whether by time, acidity or solution, frequency of change-out of solutions, or any combination) resulting in a more demineralized, more flexible material.

Thus, an implant or implant region may be partially demineralized, wherein some minerals remain and there is a range of flexibility. Alternately, an implant or implant region may be fully demineralized, wherein the minerals are basically removed and there is a maximum flexibility. As noted, during the demineralization of one region of a MCS, a transition zone may occur between the region being demineralized and an adjacent region of mineralized bone material.

Thus, an allograft segment, whether formed by either of the means described above for FIG. 28, may be comprised of one or more fully mineralized regions in combination with one or more partially demineralized regions, or with one or more fully demineralized regions, or with a combination of partially and fully demineralized regions. The arrangement in FIG. 28 is not meant to be limiting, but merely illustrative of the concept of forming or assembling two or more regions or two or more types of allografts (mineralized, partially demineralized, fully demineralized) into a single allograft segment. Thus, a wide variety of geometric arrangements may be made or assembled.

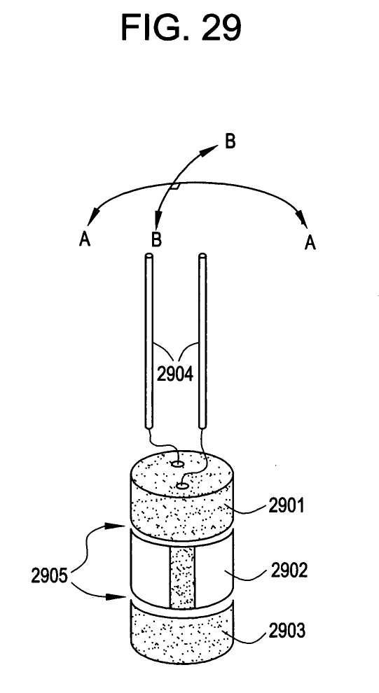

An allograft segment as described above can be combined with other allograft segments as exemplified in FIG. 29. FIG. 29 shows a first segment, 2901, that is fully mineralized, and a second segment, 2903, that is also fully mineralized. Positioned between these segments is a mixed allograft segment, 2902, such as described above in FIG. 28. Two pins, 2904, are used to secure the three segments together. Once assembled, this allograft assembly can be used in a patient in need of a degree of flexibility in the A-A dimension. Such flexibility is provided largely by the flexibility of the partially or fully demineralized side regions of the mixed-composition allograft segment, 2904. Additional flexibility may be provided by the flexibility of the pins, 2904, and the spacing between the segments, 2905.

This flexibility is advantageous post-operatively by reducing potential areas of high compression between an allograft implant and adjacent autologous bone structures. Another potential advantage for certain procedures and implants, the region(s) of demineralized or partially demineralized may remodel more rapidly and/or more strongly than the region(s) of mineralized bone. The mineralized bone region(s), however, provide structural support to transfer load during the remodeling of the demineralized or partially demineralized region(s).

Also, as described in U.S. Pat. No. 6,090,998 and its daughter applications, demineralized or partially demineralized areas of an implant may provide flexibility that is used to simulate joint flexibility.

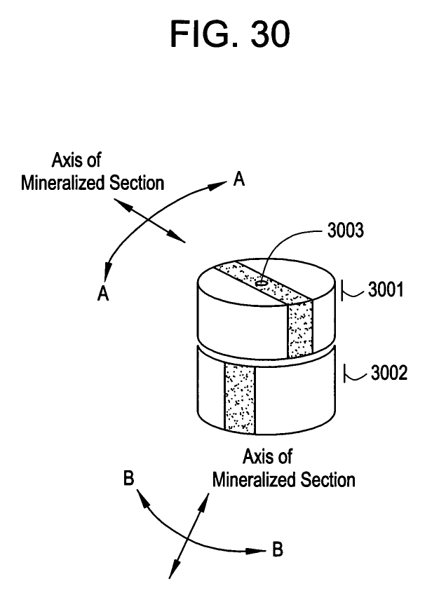

It is further noted that the present invention provides for fabrication of implants having specific, even complex, patterns of flexibility or “shock-absorbing” characteristics based on the use of MCS and/or AMCS positioned at specific orientations to other segments of an assembled allograft and to the structure in the patient in whom the implant is implanted. One example of this is depicted in FIG. 30. An assembled allograft, 3000, comprises two MCS, 3001 and 3002, which are oriented approximately 60 (and approximately 120, from a second aspect) degrees apart in relation to one another. The first MCS, 3001, permits shock absorption in the plane defined by A-A, and the second MCS, 3002, permits shock absorption in the plane defined by B-B. This allows for complex shock absorption/flexibility patterns. MCSs 3001 and 3002 are attached by a single pin connector (not shown) passing through hole 3003. The assembled allograft may include additional segments that are not MCS or AMCS, in combination with MCS or AMCS. Variations in design and construction will result from the specific requirements for an implant and the particular skill in the art as to a design or assembly means. Such variations are within the scope of the invention disclosed and claimed herein.

Regarding the assembly of an AMCS, one line of construction is to surround and/or support the separately prepared regions that are assembled together to form a segment with synthetic scaffolding. For instance, three regions, two demineralized with one mineralized region between (such as in FIG. 28) may additionally comprise a processed collagen sheet that is rolled around the assembled three regions. Also, rigid or semi-rigid synthetic structures may be used as noted above. The supplemental materials are to provide additional strength and lessen the bonding strength required on the surfaces between regions of the AMCS.

Another aspect of the invention is the use of synthetic segments and/or scaffolding in conjunction with an assembled allograft, where the assembled allograft is comprised of any combination of one or more segments each of: mineralized bone; partially demineralized bone; fully demineralized bone; or MCS or AMCS of these materials. One or more segments of assembled implants as described herein may be substituted by a synthetic segment. In addition, synthetic materials can be in the form of various scaffolding used in conjunction with one or more of the assembled segments. The synthetic segment or scaffolding may be comprised of various materials, including, but not limited to stainless steel, titanium, cobalt chromium-molybdenum alloy, and a plastic of one or more members selected from the group consisting of nylon, polycarbonate, polypropylene, polyacetal, polyethylene oxide and its copolymers, polyvinylpyrolidone, polyacrylates, polyesters, polysulfone, polylactide, and a combination of one or more bioabsorbable polymers.

In particular, biodegradable polymers suitable for use in the present invention include: poly(L-lactide) (PLLA), poly(D,L-lactide) (PLA), poly(glycolide) (PGA), poly(L-lactide-co-D,L-Lactide) (PLLA/PLA), poly(L-lactide-co-glycolide) (PLA/PGA), poly(glocolide-co-trimethylene carbonate) (PGA/PTMC), polydioxanone (PDS), polycaprolactone (PCL), polyhydroxybutyrate (PHBT), poly(phosphazenes), poly(D,L-lactide-co-caprolactone) (PLA/PCL), poly(glycolide-co-caprolactone) (PGA/PCL), poly(phosphase ester) and polyanhydrides. Other suitable materials, depending on a particular application, include hydrogens, gelatins, collagens, proteins, sodium alginate, karaya gum, guar gum, agar, algin, carrageenans, pectins, xanthan, starch based gums, hydroxyalkyl and ethyl ethers of cellulose, sodium carboxymethyl cellulose, polyvinyl alcohol, and hydrophilic polyurethanes.

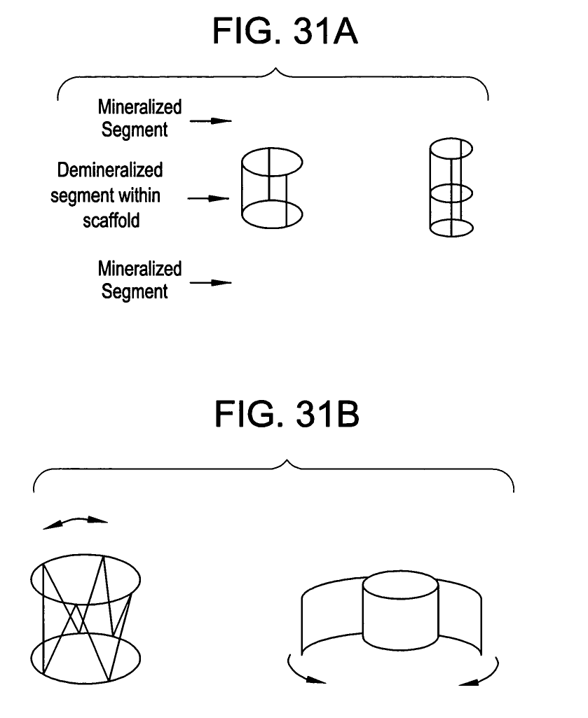

For example, a synthetic sheet may be used to wrap around a MCS or AMCS to support bone growth. Alternately, synthetic scaffolding may be rods or bars or the like, which pass through in-line holes in the respective segments. Alternately, synthetic scaffolding may be in the form of a frame that surrounds or encompasses the bulk of each segment, or the bulk of the demineralized segments, MCS, or AMCS that have flexible regions requiring structural support in the particular application in a patient in need thereof. This is employed, for instance, to add structural integrity to or around one or more segments, at least one of which has a high percentage of demineralized bone, or is otherwise in need of such additional structural support. Examples of synthetic scaffolding designs, which are not meant to be limiting, are provided in FIG. 31.

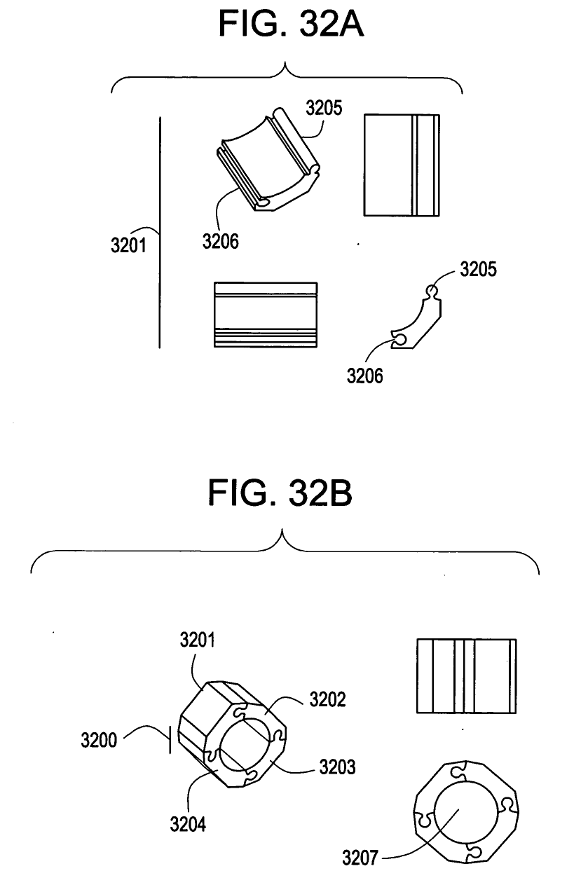

Another aspect of the invention is an assembled graft implant that is formed from at least three segments that interlock along abutting edges with one another. The shape of each segment is such that upon final assembly the major plane of each segment is non-coplanar in relation to the other segments, e.g., the segments do not lie parallel to one another. For example, FIG. 32 shows a four-piece assembled graft implant, 3200, forming a roughly circular shape. This is made up of segments 3201, 3202, 3203, and 3204. Each segment has a male edge, 3205, and a female edge, 3206, which are designed to mate with an adjoining edge. One male edge slides into a female edge of an adjacent segment, and this process continues for other edges to complete a desired assembly. When the joints of the edges interlock, as shown in FIG. 32, the joints hold the segments together.

In a preferred embodiment, assembling three or more segments results in the formation of a central channel. A central channel, 3207, is shown in FIG. 32. A central channel can be filled with osteogenic material, or may serve other purposes.

The interlocking edges are of shapes known by those skilled in the art to provide an interlocking joint. Examples, not meant to be limiting, of mateable joint designs (e.g., shapes where one part fits into or around the other) include hall and socket (as shown), tongue and groove, and mortise and tenon, such as a dovetail joint.

Also, where a portion of the body of the recipient has a need to remain intact (unsevered) yet there is a need to surround that portion with a structural support or to provide a protective barrier, segments of the present invention may be used, where the edges do not truly interlock, as defined above, but have sufficient-tolerance to permit the direct insertion of the male edge into the female edge, at once along the edges, rather than sliding from one end. This facilitates the assembly around the portion in need of structural support or protection. Optionally, one or more bands of resilient material are wrapped around the assembled structure to increase rigidity, and/or other means known in the art can be used to increase the bonding at the interlocking junctions (synthetic adhesives, bone paste, screws).

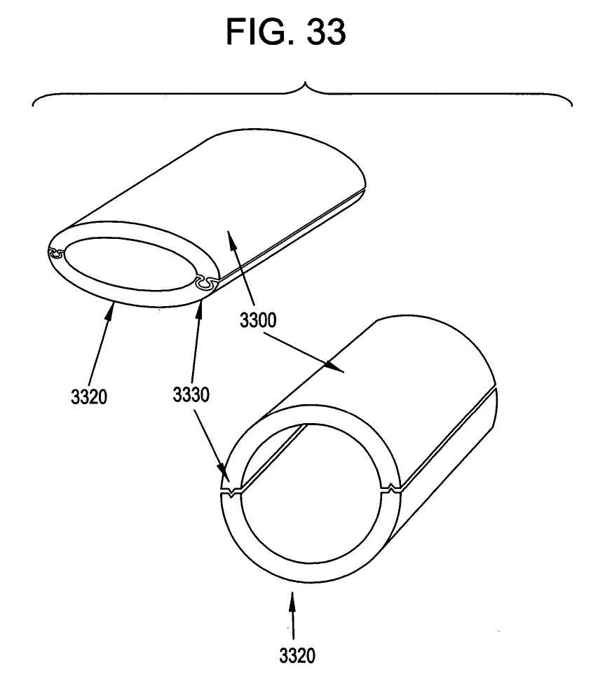

Another interlocking embodiment is two arcuate shaped segments, each having two edges of opposing interlocking edges. The edges are interlocked to form a circular or truncated circular shape, preferably with a central channel within. When the arcuate shape is a semicircle, the assembled graft is a circular. Examples, not meant to be limiting, are shown in FIG. 33, wherein segment 3300 is interlocked with segment 3310 thereby forming a channel 3320. The two embodiments shown comprise different interlocking configurations 3330.

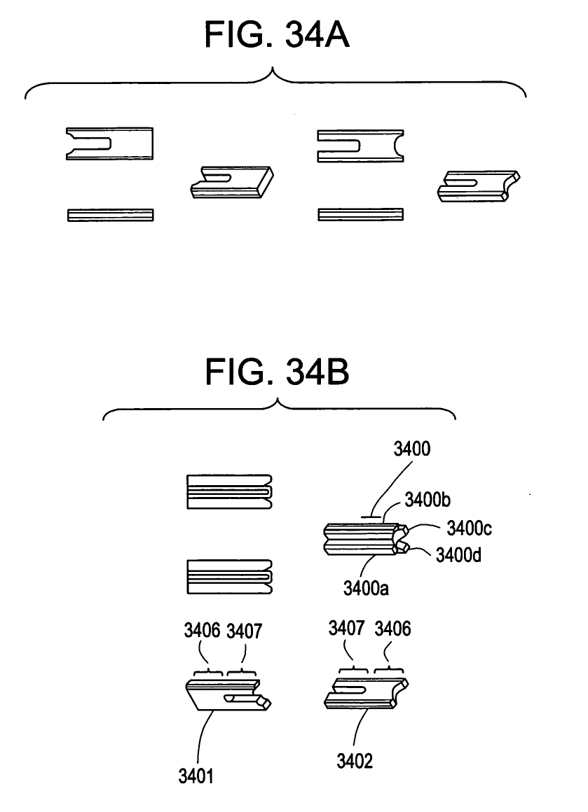

Referring to FIG. 34, another interlocking embodiment of an assembled allograft is shown as 3400, whose final cross-sectional shape is a ‘tee-’ or ‘cross’. The embodiment comprises at two individual segments 3401 and 3402 that comprise a slot 3405 longitudinally defined thereon. Thus, the segments comprise a body portion 3406 and a slotted portion 3407. When the segments are assembled they form a bone block by interlocking pieces 3401 and 3402 together. As shown, the assembled implant presents four fins, 3410a-d, that radiate from a center point, 3403. The preferred length of the assembled allograft, 3400, is approximately 2.5 mm, and the preferred diameter may range from approximately 2.0 to 12.0 mm. This assembled allograft is used for various applications where bone blocks are used. Preferably, embodiment 3400 is used in conjunction with bone-tendon-bone grafts. When used in bone-tendon or bone-tendon bone applications, preferably two separate flexible bands (natural or synthetic) are looped over the top of the embodiment 3400 wherein one band contacts fins 3410a and c, and the second band contacts fins 3410b and d. When the bone block 3400 is positioned into a channel, such as a bone tunnel formed in a patient, the two bands are compressed against the fins 3410a-d and thereby secured into place. Alternatively, the ends of the fins can comprise teeth or are otherwise irregular to further prevent slippage of the bands.

The interlocking segments described above may be made of cortical bone, cancellous bone, or a combination of cortical and cancellous bone. The segments may be of allograft or xenograft material, and preferably is treated to reduce antigenicity. In accordance with the requirements of the application, the interlocking segments are mineralized, demineralized, mixed-composition, synthetic, or a combination of these. Synthetic materials, such as those described above, may also be used in forming a segment, and alternately, in contributing to the connection of the segments in addition to the interlocking edges.

Based on the present disclosure, those skilled in the art will further appreciate that the cortical bone pins disclosed herein may have features defined thereon for various applications. For example, not meant to be limiting, the shafts may contain stops, such that other pieces of bone inserted thereon can only travel a certain distance down the shaft before encountering the stop. The shaft may also contain through holes, to permit insertion of cotter pins or the like. Furthermore, the cortical bone shaft may be demineralized, mineralized, or partially demineralized. In one specific embodiment, the end of the cortical shaft contains a tapped cannulation a short distance into the longitudinal end of the shaft. In this way, a screw may be driven into the cannulation to retain elements inserted over the shaft in association with the shaft. To accommodate the screw, the screw end bearing the cannulation may be partially demineralized, such that upon insertion of the retention screw, the shaft end does not shatter, but expands to accommodate the increasing diameter of the screw as it is driven into the shaft. Naturally, in certain applications, it may be desirable for the cortical pins to be cannulated throughout the longitudinal length thereof. However, care should be taken that this does not unduly weaken the overall compressive or torsional strength of the assembled implant. This may be addressed by including pins that are not cannulated, along with pins that are cannulated. The cannulated pins may be used in combination with sutures or the like, in order to hold an implant in a specific orientation, until fusion with adjacent bone has proceeded to a sufficient extent for the implant to become stable without the sutures.

It will be appreciated from the present disclosure that implants that have classically been fabricated from metals may be fabricated by assembling bone pieces. In addition, a benefit of the assembled graft according to this invention is that the components of the assembled graft can be derived from various anatomical structures, thus circumventing limitations normally resulting from having to obtain a graft from a particular anatomical source of a particular donor. Not only can the components be sourced from different anatomies, but also different donors may yield various components for assembly into a unitary implant. The end result is maximization of the gift of donation and the preservation of precious tissue resources. As noted above, being able to pool tissues from different sources depends, to some significant extent, on the ability to treat portions of tissue harvested from different anatomies or donors so as to prevent any contamination of a recipient with pathological or antigenic agents. A further benefit of the present invention is that different implants with height or width limitations due to the anatomical structures from which the implant has been derived may be pinned together to form implants of essentially any desired dimensions. In this fashion, an inventory of building blocks in combination with the appropriate assembly pins, threaded or unthreaded, is useful to provide implants of essentially any dimensions in the course of given surgical procedure. According to this embodiment of the invention, for example, a cervical Smith-Robinson (CSR) of any desired height may be produced by attaching two or more existing CSR implants together with cortical bone pins. This is accomplished preferably using two machined CSR's of known height such that when added together, the desired overall height is achieved. The two CSR's are stacked and drill holes are machined through the CSR bodies, following which the cortical bone pins are press-fit through the thus machined holes. Preferably, the diameter of the pins is slightly greater than the diameter of the drilled holes, such that a tight press-fit is achieved.

From the present disclosure, it will further be appreciated that implants according to this invention may be assembled in the operating room by a surgeon, using pre-formed implant pieces, from a kit. It will further be appreciated that the assembled implant pieces may be adhered to each other using any of a number of biologically acceptable glues, pastes and the like. In one such embodiment, the assembled implant pieces are assembled using a polymethyl-methacrylate glue, a cyanoacrylate glue, or any other adhesive known in the art, so long as the use of such an adhesive is confirmed to be non-toxic. It will further be appreciated that in forming the assembled grafts according to the present invention, it is acceptable, although not required, for interlocking features to be included on abutting faces of implant segments to be assembled together. Where such features are included, it is preferred for the adjacent features to be complementary, such that a protrusion on a first surface is met by a compatible indentation in the abutting surface. Such abutting features assist to provide torsional and structural strength to the assembled implant, and to relieve a measure of stress on the cortical bone pins used to assemble the implant.

According to U.S. Pat. No. 6,025,538, an elaborate system is disclosed for ensuring that a bore is provided in mating surfaces of a composite implant such that the bore is angularly aligned with respect to mating surfaces so as to be oblique to the plane of each mating surface. This is not required according to the present invention.

According to U.S. Pat. No. 5,899,939, layers of bone are juxtaposed, but no mechanical fixation of the various layers to each other is provided for, such as the cortical bone pins disclosed herein.

Having generally described this invention, including the methods of manufacture and use thereof, including the best mode thereof, those skilled in the art will appreciate that a large number of variations on the principles described herein may be accomplished.

Thus, the specifics of this description and the attached drawings should not be interpreted to limit the scope of this invention to the specifics thereof. Rather, the scope of this invention should be evaluated with the reference to the claims appended hereto.

What is claimed is:

1. A method for manufacture of autograft, allograft and xenograft implants which comprises assembling such implants from smaller pieces of graft materials to form a larger graft implant product.

2. A kit comprising assemblable parts of autograft, allograft and xenograft implants for assembling such implants from smaller pieces of graft materials to form a larger graft implant product which may be formed in the course of a surgical procedure to precisely meet the needs of a given patient or procedure.

3. A method of strengthening or reinforcing autograft, allograft and xenograft implants which comprises assembling such implants from smaller pieces of graft materials to form a larger graft implant product.

4. The method of claim 3 wherein the reinforced product is cancellous bone into which is inserted reinforcing material.

5. The method according to claim 4 wherein said reinforcing material comprises cortical bone.

6. A graft implant comprising any one or combinations of allograft materials, autograft materials, xenograft materials, synthetic materials, metallic materials assembled into a an assembled implant which is assembled into a single graft by use of reinforcing material to hold the constituent pieces of graft materials together.

7. The graft implant according to claim 6 wherein said reinforcing material comprises cortical bone.

8. The graft implant according to claim 6 wherein said any one or combinations of allograft materials, autograft materials, xenograft materials, synthetic materials, metallic materials are pretreated by a process comprising removing associated non-bone adventitious materials from a bone graft to provide a cleaned bone graft, contacting the cleaned bone graft with defatting solutions to provide a cleaned defatted bone graft, and contacting said cleaned defatted bone graft with a chaotropic agent to remove non-collagenous or non-structural collagen proteins.

9. The graft implant according to claim 8 wherein said chaotropic agent is selected from urea, guanidinium hydrochloride, Tween, TritonX-100, TNBP, SDS, and mixtures of these agents.

10. The graft implant according to claim 6 wherein said any one or combinations of allograft materials, autograft materials, xenograft materials, synthetic materials, metallic materials are pretreated by a process comprising cleaning, perfusion and passivation process which comprises cyclic exposure of said implant to increased and decreased positive or negative pressures, or both.

11. The graft implant according to claim 10 wherein a cleaning solution used during the cleaning step is selected from the group consisting of: sterile water, Triton X-100, TNBP, 3% hydrogen peroxide, a water-miscible alcohol, saline solution povidone iodine, ascorbic acid solution, aromatic or aliphatic hydrocarbons, ethers, ketones, amines, urea, guanidine hydrochloride, esters, glycoproteins, proteins, saccharides, enzymes, gasseous acids or peroxides, and mixtures thereof.

12. The graft implant according to claim 6 wherein the assembled implant is pre-treated or treated after assembly to incorporate biologically active or inert materials.

13. An implant comprising segments of cortical bone, cancellous bone, cortical-cancellous bone, or combinations thereof pinned to each other by means of cortical bone pins, wherein, prior to assembly or after assembly, the graft materials are soaked, infused, impregnated, coated or otherwise treated with bone morphogenetic proteins (BMP's), antibiotics, growth factors, nucleic acids, peptides, or combinations thereof.

14. The implant according to claim 6 comprising an assembled cancellous block, or dowel, harvested from the iliac crest or another suitable site to form a Cloward Dowel, iliac crest wedge, or cancellous bone block, dowel, reinforced by insertion therein of cortical bone pins.

15. The implant according to claim 6 comprising a cortical bone implant reinforced by insertion therein of at least one cortical bone pin.

16. The implant according to claim 6 comprising an assembled implant comprising different segments of cortical bone, cancellous bone or both.

17. The implant according to claim 6 comprising an assembled implant comprising different segments of cortical bone, cancellous bone, demineralized cortical or cancellous bone, synthetic material, and combinations thereof.

18. The implant according to claim 17 wherein insertion of reinforcing pins provides an implant with multiple load-bearing pillars.

19. The implant according to claim 18 wherein said pins protrude from the surface of the implant to engage with inferior, superior or both surfaces of bone between which the implant is inserted.

20. The implant according to claim 19 which is a spinal implant.

21. The implant according to claim 19 comprising a cancellous portion of bone implant that has been compression molded, and then affixed to other portions of cortical or cancellous bone machined according to different or similar principles.

22. The implant according to claim 6 in the form of a tapered dowel.

23. A method of repairing a bone implant which comprises insertion therein of at least one cortical bone pin.

24. The method according to claim 23 which further comprises affixing a piece of bone to an existing bone implant by affixing said piece of bone to said cortical bone pin.

25. The method according to claim 1 for making an instrument for insertion of other implants.

26. The method according to claim 24 which is an implant driver.

27. A method for salvaging an implant that does not meet manufacturing specifications which comprises insertion of at least one cortical bone pin at a site to reinforce said site such that in combination with said at least one cortical bone pin, said implant meets manufacturing specifications.

28. An assembled implant comprising a first bone segment pinned to a second bone segment with a flexible tissue affixed between said first bone segment and said second bone segment

29. The assembled implant according to claim 28 wherein said first and second bone segments are affixed to each other by means of at least one cortical bone pin.

30. An assembled graft implant comprising two or more individual segments fastened together, said implant comprising at least one demineralized bone segment and at least one mineralized bone segment.

31. The assembled graft implant of claim 30, wherein said at least one demineralized bone segment comprises a region of mineralized bone.

32. The assembled graft implant of claim 30, wherein said demineralized or mineralized segments are made from cortical bone, cancellous bone or both.

33. An assembled graft implant comprising two or more individual segments fastened together, said implant comprising at least one synthetic segment and at least one demineralized bone segment.

34. The assembled graft implant of claim 33, wherein said demineralized bone segment comprises a region of mineralized bone.

35. The assembled graft implant of claim 33, wherein said synthetic segment is comprised of stainless steel, titanium, cobalt chromium-molybdenum alloy, nylon, polycarbonate, polypropylene, polyacetal, polyethylene oxide and its copolymers, polyvinylpyrolidone, polyacrylates, polyesters, polysulfone, polylactide, poly(L-lactide) (PLLA), poly(D,L-lactide) (PLA), poly(glycolide) (PGA), poly(L-lactide-co-D,L-Lactide) (PLLA/PLA), poly(L-lactide-co-glycolide) (PLA/PGA), poly(glocolide-co-trimethylene carbonate) (PGA/PTMC), polydioxanone (PDS), polycaprolactone (PCL), polyhydroxybutyrate (PHBT), poly(phosphazenes), poly(D,L-lactid-co-caprolactone) (PLA/PCL), poly(glycolide-co-caprolactone) (PGA/PCL), poly(phosphase ester), polyanhydrides, polyvinyl alcohol, hydrophilic polyulrethanes, and a combination of one or more bioabsorbable polymers.

36. The assembled graft implant of claim 33, wherein said at least one synthetic segment comprises a first end and a second end, and wherein a demineralized bone segment or a mineralized bone segment is attached to said first end or said second end.

37. An assembled graft implant comprising two or more individual segments fastened together, said implant comprising at least one synthetic segment and at least one mineralized bone segment.

38. The assembled graft implant of claim 37, wherein said synthetic segment is comprised of stainless steel, titanium, cobalt chromium-molybdenum alloy, and a plastic of one or more members selected from the group consisting of nylon, polycarbonate, polypropylene, polyacetal, polyethylene oxide and its copolymers, polyvinylpyrolidone, polyacrylates, polyesters, polysulfone, polylactide, poly(L-lactide) (PLLA), poly(D,L-lactide) (PLA), poly(glycolide) (PGA), poly(L-lactide-co-D,L-Lactide) (PLLA/PLA), poly(L-lactide-co-glycolide) (PLA/PGA), poly(glocolide-co-trimethylene carbonate) (PGA/PTMC), polydioxanone (PDS), polycaprolactone (PCL), polyhydroxybutyrate (PHBT), poly(phosphazenes), poly(D,L-lactide-co-caprolactone) (PLA/PCL), poly(glycolide-co-caprolactone) (PGA/PCL), poly(phosphase ester), polyanhydrides, polyvinyl alcohol, hydrophilic polyurethanes, and a combination of one or more bioabsorbable polymers.

39. An assembled graft implant comprising two or more individual segments fastened together, wherein said assembled graft comprises at least one segment comprised of demineralized bone, mineralized bone, demineralized bone having a mineralized region, or a synthetic material, and at least one other segment fastened thereto that is comprised of demineralized bone, mineralized bone, demineralized boric having a mineralized region, or a synthetic material.

40. A graft segment configured for assembly with at least one other segment, wherein said graft segment comprises at least one mineralized bone region and at least one demineralized bone region.

41. The graft segment of claim 40, wherein said mineralized bone region is attached to or integrated with said demineralized bone region.

42. A graft segment according to claim 40, wherein said graft segment comprises a central mineralized bone region and at least one demineralized bone region integrated with said central mineralized bone region and positioned on one or more sides of or surrounding said mineralized bone region.

43. A mixed composition segment configured for assembly with at least one other segment, said mixed composition segment comprising a region comprised of mineralized bone, demineralized bone or a synthetic material that is attached to or integrated with another region comprised of mineralized bone, demineralized bone or a synthetic material.

44. The mixed composition segment of claim 43, additionally assembled with at least one other graft segment.

45. A method for manufacture of a mixed-composition segment for autograft, allograft and xenograft graft implants comprising contacting a region of a mineralized bone segment with a demineralizing solution for a period of time sufficient to achieved a desired level of demineralization to said region.

46. The method of claim 45 further comprising removing a sufficient quantity of said demineralizing solution from said first region to prevent a toxic or an inflammatory response to said segment upon implantation into a patient in need thereof.

47. The method of claim 46, wherein said contacting is repeated for at least one additional region, and said removing step is done to said at least one additional region at the same time or at a different time as for said first region.

48. A mixed-composition segment produced by the method of claim 45.

49. A mixed-composition segment produced by the method of claim 45, wherein at least one region of said mixed-composition segment is mineralized bone, and at least one region of said mixed-composition segment is demineralized bone.

50. A mixed-composition segment produced by the method of claim 45, wherein one region of said mixed-composition segment is mineralized, and one or more regions of said mixed-composition segment are demineralized, wherein said one or more regions surround or sandwich said region of mineralized bone.

51. A method for manufacture of a mixed-composition segment for autograft, allograft and xenograft graft implants comprising
a. contacting a first piece of graft material comprising bone with a demineralizing solution for a period of time sufficient to achieve a desired level of demineralization to said first piece; and
b. bonding or otherwise intimately attaching a portion (region) of said first piece of demineralized graft material with a second piece of graft material, said second piece of graft material being mineralized, demineralized, or synthetic, such that said bonding or intimately attaching results it a single integral mixed-composition segment; and
c. optionally, removing a sufficient quantity of said demineralizing solution from said first region to prevent a toxic or an inflammatory response to said segment upon implantation into a patient in need thereof.

52. The method of claim 51, wherein step (a) is repeated for at least one additional piece, and step (b) is repeated to attach each at least one additional piece to form a multi-piece (multi-region) mixed-composition segment.

53. A mixed-composition segment produced by the method of claim 51.

54. A mixed-composition segment produced by the method of claim 51, wherein at least one region of said mixed-composition segment is mineralized bone, and at least one region of said mixed-composition segment is demineralized bone.

55. A mixed-composition segment produced by the method of claim 51, wherein one region of said mixed-composition segment is mineralized bone, and one or more regions of said mixed-composition segment are demineralized bone, wherein said demineralized bone regions surround or sandwich said region of mineralized bone.

56. A kit comprising assemblable parts of autograft, allograft, xenograft and synthetic segments for assembling mixed-composition implants from smaller pieces of graft materials to form a larger graft implant product which may be formed in the course of a surgical procedure to precisely meet the needs of a given patient or procedure, and comprising at least one mixed-composition segment among said assemblable parts.

57. A method of strengthening or reinforcing a mixed-composition segment for autograft, allograft and xenograft graft implants which comprises assembling said mixed-composition segment from smaller pieces of graft materials to form a larger mixed-composition segment.

58. The method of claim 57 wherein said mixed-composition segment comprises cancellous bone in combination with demineralized bone.

59. The method of claim 57 wherein the mixed-composition segment comprises cortical bone in combination with demineralized bone.

60. An implant comprising segments of cortical bone, cancellous bone, cortical-cancellous bone, or combinations thereof pinned to each other by means of cortical bone pins, wherein, prior to assembly or after assembly, the graft materials are soaked, infused, impregnated, coated or otherwise treated with bone morphogenetic proteins (BMP's), antibiotics, growth factors, nucleic acids, peptides, sodium hyaluronate, hyaluronic acid, polysulfated glycosaminoglycans, or combinations thereof, and wherein, at least one of said segments is a mixed-composition segment or demineralized bone.

61. An assembled implant comprising a first bone segment pinned to a second bone segment, and comprising a flexible tissue affixed between said first bone segment and said second bone segment, wherein said first bone segment is a mixed-composition segment.

62. An assembled implant bone graft comprising at least two individual segments joined together, and synthetic scaffolding material, wherein said synthetic scaffolding material passes through and/or surrounds said segments, thereby providing structural support to at least one of said at least two individual segments.

63. An assembled bone graft comprising:
a. a first graft segment comprising at least one mineralized bone region, and at least one demineralized bone region; and comprising at least one hole;
b. at least one other graft segment comprising at least one hole; and
c. at least one connector;
d whereby the first graft segment and the at least one other graft segment are coined physically by said at least one connector.

64. The bone graft of claim 63, wherein said first graft segment and said at least one other graft segment are joined physically by means of at least one pin, rod, bar, post or other linear connector passing through said at least one hole in said first graft segment which is arranged to align with said at least one hole of said other graft segment.

65. The bone graft of claim 63, additionally comprising a synthetic support structure that encompasses all or a part of said composite bone graft whereby the synthetic support structure bears load that would otherwise bear on at least one of said graft segments.

66. The bone graft of claim 65, wherein said synthetic support structure is comprised of a biocompatible material selected from the group consisting of stainless steel, titanium, cobalt chromium-molybdenum alloy, and a plastic of one or more members selected from the group consisting of nylon, polycarbonate, polypropylene, polyacetal, polyethylene oxide and its copolymers, polyvinylpyrolidone, polyacrylates, polyesters, polysulfone, polylactide, poly(L-lactide) (PLLA), poly(D,L-lactide) (PLA), poly(glycolide) (PGA), poly(L-lactide-co-D,L-Lactide) (PLLA/PLA), poly(L-lactide-co-glycolide) (PLA/PGA), poly(glocolide-co-trimethylene carbonate) (PGA/PTMC), polydioxanone (PDS), polycaprolactone (PCL), polyhydroxybutyrate (PHBT), poly(phosphazenes), poly(D,L-lactide-co-caprolactone) (PLA/PCL), poly(glycolide-co-caprolactone) (PGAIPCL), poly(phosphase ester), polyanhydrides, polyvinyl alcohol, hydrophilic polyurethanes, and a combination of one or more bioabsorbable polymers.

67. A graft implant comprising any one or combinations of allograft materials, autograft materials, xenograft materials, synthetic materials, and metallic materials assembled into an assembled implant which is assembled into a single graft by use of reinforcing material to hold the constituent pieces of graft materials together, and comprising at least one mixed-composition segment.

68. The graft implant of claim 67 wherein said reinforcing material comprises cortical bone.

69. The graft implant of claim 67 wherein the assembled implant is pre-treated or treated after assembly to incorporate biologically active or inert materials.

70. The implant of claim 67 comprising an assembled cancellous block, or dowel, harvested from the iliac crest or another suitable site to form a Cloward Dowel, iliac crest wedge, or cancellous bone block, dowel, reinforced by insertion therein of cortical bone pins.

71. The implant of claim 67 comprising a cortical bone implant reinforced by insertion therein of at least one cortical bone pin.

72. The implant of claim 67 comprising an assembled implant comprising different segments of cortical bone, cancellous bone or both.

73. The implant of claim 67 in the form of a tapered dowel.

74. The implant of claim 67 comprising an assembled implant comprising different segments of cortical bone, cancellous bone, demineralized cortical or cancellous bone, or synthetic material, or combinations thereof.

75. The implant of claim 71 wherein insertion of reinforcing pins provides an implant with multiple load-bearing pillars.

76. The implant of claim 75 wherein said pins protrude from the surface of the implant to engage with inferior, superior or both surfaces of bone between which the implant is inserted.

77. The implant of claim 67 which is a spinal implant.

78. The implant according to claim 67 comprising a cancellous portion of bone implant that has been compression molded, and then affixed to other portions of cortical or cancellous bone machined according to different or similar principles.

79. A bone implant comprising:
a. two or more bone segments,
b. at least one biocompatible connector,
c. wherein said at least one biocompatible connector fastens together said two or more bone segments to form an assembled bone implant, said at least one biocompatible connector does not comprise an adhesive.

80. The bone implant of claim 79, wherein at least one of said two or more bone segments is a mixed composition segment.

81. An assembled bone graft comprising at least three segments, each said segment comprising a first edge and a second edge at a side opposite from the first edge, the first and second edges having interlocking structures mateable with an adjacent edge of an adjacent segment, whereby each said segment's first and second edges interlock with the edges of adjacent segments.

82. An assembled bone graft comprising at least three non-coplanar segments, each said segment comprising a first mateable edge and a second mateable edge, each of said mateable edges being mateable with an adjacent mateable edge of an adjacent segment, whereby said assembled bone graft is assembled by mating said first edges and said second edges of said segments positioned adjacent to one another.

83. The assembled bone graft of claim 82, wherein said mateable edges interlock, and are selected from the group of joint types consisting of ball and socket, tongue and groove, and mortise and tenon.

84. The assembled bone graft of claim 82, additionally comprising at least one band of flexible, non-stretchable material wrapped around the circumference of said assembled bone graft.

85. The assembled bone graft of claim 82, wherein at least one of said segments is comprised of a material selected from the group consisting of demineralized bone, mineralized bone, a combination of demineralized and mineralized bone.

86. The assembled bone graft of claim 82, wherein at least one of said segments is comprised of a material selected from the group consisting of cortical bone, cancellous bone, and a combination of cortical and cancellous bone.

87. The assembled bone graft of claim 82, wherein at least one of said segments is comprised of any one or combinations of allograft materials, autograft materials, xenograft materials, synthetic materials, and metallic materials assembled into a segment.

88. An assembled bone graft comprising a first and a second arcuate-shaped segment, each segment comprising two interlocking edges, whereby each said edge of said first segment interlocks with an edge of said second segment, forming an assembled bone graft with an open channel between said first and second segments.

89. A bone tendon bone-type graft useful in orthopedic surgery comprising at least one block and a flexible band attached to said at least one block.

90. The bone tendon bone-type graft of claim 89, wherein at least one block of the at least one block is comprised of a synthetic material, and the flexible band is comprised of allograft or xenograft tendon, ligament, or processed dermis.

91. The bone tendon bone-type graft of claim 89, wherein at least one of the at least one block is comprised of cortical bone, cancellous bone, cortico-cancellous bone, or a combination of these, and the flexible band is comprised of a synthetic material.

92. The bone tendon bone-type graft of claim 91, wherein said synthetic material is comprised of a biocompatible material selected from the group consisting of nylon, polycarbonate, polypropylene, polyacetal, polyethylene oxide and its copolymers, polyvinylpyrolidone, polyacrylates, polyesters, polysulfone, polylactide, poly(L-lactide) (PLLA), poly(D,L-lactide) (PLA), poly(glycolide) (PGA), poly(L-lactide-co-D,L-Lactide) (PLLA/PLA), poly(L-lactide-co-glycolide) (PLA/PGA), poly(glocolide-co-trimethylene carbonate) (PGA/PTMC), polydioxanone (PDS), polycaprolactone (PCL), polyhydroxybutyrate (PHBT), poly(phosphazenes), poly(D,L-lactide-co-caprolactone) (PLA/PCL), poly(glycolide-co-caprolactone) (PGA/PCL), poly(phosphase ester), polyanhydrides, polyvinyl alcohol, and hydrophilic polyurethanes.

93. The bone tendon bone-type graft of claim 91, wherein at least one of the at least one block is comprised of an assembled bone graft.

94. The bone tendon bone-type graft of claim 92, wherein the assembled bone graft is comprised of at least one mixed-composition segment.

95. A method of assembling an assembled implant to obtain a desired interference fit, comprising:
a. vacuum drying at least one bone pin to obtain a desired size reduction;
b. measuring the diameter of the at least one bone pin after vacuum drying;
c. making at least one hole in at least one bone piece to be assembled with the at least one bone pin, wherein the hole is smaller than the diameter of the at least one hone pin to obtain an interference fit;
d. assembling the at least one bone pin with the at least one bone piece by inserting each of the at least one pin(s) through the at least one hole(s) to form the assembled implant; and
e. freeze drying the assembled implant;
whereby the interference fit(s) between the at least one bone pin and the at least one hole in the at least one bone piece fall within a desired range.

96. The method of claim 95 wherein the at least one bone pin is comprised of cortical bone, and optionally at least one of the at least one bone piece is comprised of cortical bone.

97. The method of claim 96 wherein the desired range for the interference fit is 0.001 to 0.003 inches.

98. The method of claim 96 wherein the vacuum drying is at room temperature, is conducted at a negative pressure of approximately 100 milliTorre, and lasts at least five hours.

99. An assembled implant comprising at least two substantially planar segments, wherein at least one of said at least two substantially planar segments comprise at least one slot defined thereon, and wherein said at least two substantially planar segments are fastened together by sliding said at least one slot of at least one planar segment over another substantially planar segment.

100. The assembled implant of claim 99, said implant comprising a first substantially planar segment and a second substantially planar segment, wherein said first and second substantially planar segments comprise a slot longitudinally defined thereon such that said first and second substantially planar segments comprise a slotted section and a body section, and wherein said first and second substantially planar segments are fastened together by sliding the slotted section of each over the body portion of the other.

101. A bone-tendon graft comprising at least one assembled bone block, wherein said bone block is comprised of mineralized bone, demineralized bone or a sythetic material, or a mixed composition; and at least one flexible band attached to said at least one bone block, wherein said band is comprised of demineralized bone or of a synthetic material.
